(19) United States
(12) Patent Application Publication
(10) Pub. No.: US 2025/0320487 A1
(43) Pub. Date: Oct. 16, 2025

(54) Preparation Of Size-Controlled Nucleic Acid Fragments

(71) Applicant: Illumina, Inc., San Diego, CA (US)

(72) Inventors: Niall Anthony Gormley, Cambridge (GB); Andrew Slatter, Cambridge (GB); Stephen Gross, San Diego, CA (US); Kayla Busby, San Diego, CA (US); Allison Yunghans, San Diego, CA (US); Morgan Roos, Middleton, WI (US); Robert Scott Kuersten, Madison, WI (US)

(21) Appl. No.: 18/865,132

(22) PCT Filed: May 17, 2023
(86) PCT No.: PCT/US2023/022553

§ 371 Date: Nov. 12, 2024

Related U.S. Application Data
(60) Provisional application No. 63/343,261, filed on May 18, 2022.

(51) Int. Cl. C12N 15/10

(52) U.S. Cl. CPC C12N 15/1068; C12N 15/1065

(57) ABSTRACT

A transposome complex capable of producing size-controlled nucleic acid fragments is described herein. In some embodiments, the transposome complex includes multiple inactive transposomes with active transposomes on both ends of the multiple inactive transposomes. Applications, uses, and variations of the disclosed transposome complex include, but are not limited to, library preparation for a nucleic acid and tuning the length of the transposome complex to produce nucleic acid fragments of predetermined or desired lengths.

### BACKGROUND

The technology disclosed relates to nucleic acid sequencing. In particular, the technology disclosed relates to a transposome complex that can be used to generate size-controlled nucleic acid fragments, e.g., as part of generating a sequencing library for nucleic acid sequencing.

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Sample preparation (e.g., library preparation) for next-generation sequencing can involve fragmentation of nucleic acids, such as genomic DNA or double-stranded cDNA (prepared from RNA) into smaller fragments, followed by addition of functional tag sequences (“tags”) to the strands of the fragments. Such tags include priming sites for DNA polymerases for sequencing reactions, restriction sites, and domains for capture, amplification, detection, address, and transcription promoters. Previous methods for generating DNA fragment libraries may involve fragmenting the target DNA mechanically using a sonicator, nebulizer, or by a nuclease, and then joining (e.g., by ligation) the oligonucleotides containing the tags to the ends of the fragments.

The use of transposomes, protein-DNA complexes of a transposase and transposon sequences that tag and fragment (“tagment”) DNA by transposition, allows for simultaneous genomic fragmentation and adaptor incorporation into fragments, thereby simplifying library preparation. A method for using transposomes to rapidly achieve these steps was disclosed in US 2010/0120098 by Grunenwald, which is incorporated herein by reference for all purposes, to generate fragments from any double-stranded DNA (e.g. genomic, amplicon, viral, phage, cDNA derived from RNA, etc.). Transposon systems include the hyperactive Tn5 transposon system described in U.S. Pat. Nos. 5,965,443 and 6,437,109 by Reznikoff, and the Mu transposon system in U.S. Pat. No. 6,593,113 by Tenkanen, all of which are incorporated herein by reference. Reznikoff described a 19-base transposase end sequence that is frequently referred to as “ME”, a mosaic end sequence. Transposon end tagging is used to tag nucleic acid fragments generated from a biological sample. Described herein are techniques for improving a transposon-mediated nucleic acid fragment generation process and, therefore, improving subsequent nucleic acid sequencing from such fragments.

### BRIEF DESCRIPTION

In one embodiment, the present disclosure relates to a transposome complex. The transposome complex includes a plurality of inactive transposomes coupled to one another. Each inactive transposome of the plurality of inactive transposomes includes a transposase and an oligonucleotide adaptor. The transposome complex also includes a first active transposome coupled to a first end of the plurality of inactive transposomes. Further, the transposome complex includes a second active transposome coupled to a second end of the plurality of inactive transposomes such that the plurality of inactive transposomes are positioned between the first active transposome and the second active transposome.

In another embodiment, the present disclosure relates to a method of preparing a transposome complex. The method includes providing an initiator transposome. The initiator transposome includes a transposome dimer, a first at least partially double-stranded oligonucleotide adaptor coupled to the transposome dimer, and a second at least partially double-stranded oligonucleotide adaptor coupled to the transposome dimer. The method also includes hybridizing at least one linking transposome to the initiator transposome via an at least partially double-stranded linking adaptor of the at least one linking transposome, wherein the at least partially double-stranded linking adaptor is complementary to the first at least partially double-stranded oligonucleotide adaptor, the second at least partially double-stranded oligonucleotide adaptor, or both. Further, the method includes coupling at least one terminal transposome to the at least one linking transposome via an at least partially double-stranded terminal adaptor of the terminal transposome that is complementary to the at least partially double-stranded linking adaptor or a different linking adaptor of the at least one linking transposome, wherein the terminal transposome is catalytically active and wherein the at least one linking transposome is catalytically inactive.

In another embodiment, the present disclosure relates to a method of preparing a nucleic acid library. The method includes contacting target nucleic acids with a plurality of transposome complexes. Each transposome complex of the plurality includes a first active transposome coupled to a second active transposome via an intervening plurality of inactive transposomes, to permit binding of the plurality of transposome complexes to the target nucleic acids. The method also includes tagmenting the target nucleic acids to generate nucleic acid fragments. A size of the generated nucleic acid fragments is a function of a size of an individual transposome complex of the plurality of transposome complexes.

In another embodiment, the present disclosure relates to a surface-linked transposome complex. The surface-linked transposome complex includes a surface and a plurality of transposomes coupled to the solid surface. Each transposome of the plurality of transposomes includes a transposase and an oligonucleotide adaptor. Each transposome of the plurality of transposomes is inactive based on a modification of the oligonucleotide adaptor.

In another embodiment, the present disclosure relates to a method of separating nucleic acids. The method includes contacting a plurality of transposome complexes with a mixed nucleic acid sample in solution. The mixed nucleic acid sample includes double-stranded DNA and RNA such that the double-stranded DNA selectively binds to the plurality of transposome complexes relative to the RNA. Each transposome complex of the plurality includes a plurality of transposomes coupled to a surface. Further, each transposome of the plurality of transposomes is inactive based on a modification of an oligonucleotide adaptor. The method also includes separating the double-stranded DNA from RNA by removing the plurality of transposomes complexes with bound double-stranded DNA from the solution, the solution comprising the RNA.

In another embodiment, the present disclosure relates to a method of normalizing an amount of nucleic acids for a plurality of samples. The method includes contacting a first plurality of double-stranded nucleic acids of a first sample with a first plurality of transposome complexes. Each transposome complex of the first plurality of transposome complexes includes a predetermined amount or range of transposomes coupled to a bead surface. Further, each transposome of the first plurality of transposome complexes is inactive based on a modification of an oligonucleotide adaptor. Even further, the contacting is under conditions such that a portion of the first plurality of double-stranded nucleic acids binds to the first plurality of transposome complexes. The method also includes contacting a second plurality of double-stranded nucleic acids of a second sample with a second plurality of transposome complexes. Each transposome complex of the second plurality of transposome complexes includes the predetermined amount or range of transposomes coupled to a bead surface. Each transposome of the second plurality of transposome complexes is inactive based on a modification of an oligonucleotide adaptor. Further, the contacting is under conditions such that a portion of the second plurality of double-stranded nucleic acids binds to the second plurality of transposome complexes. Further still, the method includes sequencing the bound portion of the first plurality of double-stranded nucleic acids and the bound portion of the second plurality of double-stranded nucleic acids.

In another embodiment, the present disclosure relates to a method of performing a buffer exchange. The method includes contacting a plurality of nucleic acids suspended in a first buffer solution with a plurality of transposome complexes. Each transposome complex of the plurality of transposome complexes includes a plurality of transposomes coupled to a surface. Further, each transposome of the plurality of transposomes is inactive based on a modification of an oligonucleotide adaptor. The method also includes producing a pellet comprising the plurality of nucleic acids bound to the plurality of transposome complexes. Further, the method includes separating the pellet from the first buffer solution. Further still, the method includes suspending the pellet in a second buffer solution.

The preceding description is presented to enable the making and use of the technology disclosed. Various modifications to the disclosed implementations will be apparent, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein. The scope of the technology disclosed is defined by the appended claims.

### BRIEF DESCRIPTION OF THE DRAWINGS

These and other features, aspects, and advantages of the present techniques will become better understood when the following detailed description is read with reference to the accompanying drawings in which like characters represent like parts throughout the drawings, wherein:

FIG. 1 is a diagram of a method for preparing a nucleic acid library, in accordance with aspects of the present disclosure;

FIG. 2 is a schematic diagram of an example of a transposome complex that may be utilized to generate size-controlled fragments, in accordance with aspects of the present disclosure;

FIG. 3 is a diagram of a method for preparing a library from a target nucleic acid using the transposome complex of FIG. 2, in accordance with aspects of the present disclosure;

FIG. 4A is a schematic diagram of inactive transposomes and active transposomes that may be used to generate the transposome complex of FIG. 2, in accordance with aspects of the present disclosure;

FIG. 4B is a schematic diagram of a first inactive transposome hybridizing to a second inactive transposome via respective adaptors, in accordance with aspects of the present disclosure;

FIG. 4C is a schematic diagram of a second inactive transposome hybridizing to a third inactive transposome via respective adaptors, in accordance with aspects of the present disclosure;

FIG. 4D is a schematic diagram of a third inactive transposome hybridizing to an active transposome via respective adaptors, in accordance with aspects of the present disclosure;

FIG. 5 is a diagram of a method for generating the transposome complex of FIG. 2, in accordance with aspects of the present disclosure;

FIG. 6 is a perspective view of an asymmetric transposome complex attached to a substrate, in accordance with aspects of the present disclosure;

FIG. 7 shows a plot of template insert sizes that result from sequencing DNA libraries, in accordance with aspects of the present disclosure;

FIG. 8A shows a schematic diagram of a surface-linked transposome complex (SLTC) including inactive transposomes, in accordance with aspects of the present disclosure;

FIG. 8B shows a schematic diagram of a SLTC including inactive transposomes and active transposomes, in accordance with aspects of the present disclosure;

FIG. 9 shows a graph illustrating fragment size modulation based on addition of inactive transposomes to a SLTC, in accordance with aspects of the present disclosure;

FIG. 10 shows gel electrophoresis results illustrating transposome formation, in accordance with aspects of the present disclosure;

FIG. 11 shows a first graph illustrating tagmentation activity of a bead-linked transposome complex (BLT) including active transposomes and tagmentation activity of a bead-linked transposome complex including inactive transposomes (zBLT), in accordance with aspects of the present disclosure;

FIG. 12 shows a second graph illustrating products of tagmentation treatment of a BLT including active transposomes and products of a BLT containing only inactive transposomes, in accordance with aspects of the present disclosure;

FIG. 13 shows a graph illustrating an amount of nucleic acid bound to BLTs and zBLTs, in accordance with aspects of the present disclosure;

FIG. 14 shows a first graph illustrating tagmentation activity of a BLT and tagmentation activity of a zBLT for different ratios of active and inactive transposomes, in accordance with aspects of the present disclosure;

FIG. 15A shows a first graph illustrating tagmentation activity of a zBLT with a first amount of inactive transposomes, in accordance with aspects of the present disclosure;

FIG. 15B shows a second graph illustrating tagmentation activity of a zBLT with a second amount of inactive transposomes, in accordance with aspects of the present disclosure;

FIG. 15C shows a third graph illustrating tagmentation activity of a zBLT with a third amount of inactive transposomes, in accordance with aspects of the present disclosure;

FIG. 16 shows graphs illustrating conversion efficiency, sensitivity, and mean insertion length for a control bead-linked transposomes and bead-linked transposomes including inactive transposomes, in accordance with an embodiment;

FIG. 17 is a diagram of a method for separating nucleic acids using the SLTC of FIG. 8A, in accordance with aspects of the present disclosure;

FIG. 18 is a diagram of a method for normalizing an amount of nucleic acids using the SLTC of FIG. 8A, in accordance with aspects of the present disclosure;

FIG. 19A is a diagram of a method for transferring nucleic acids from a first solution to a second solution, in accordance with aspects of the present disclosure;

FIG. 19B is a diagram of a first method for transferring nucleic acids from a first solution to a second solution using the SLTC of FIG. 8A, in accordance with aspects of the present disclosure;

FIG. 19C is a diagram of a second method for transferring nucleic acids from a first solution to a second solution using the SLTC of FIG. 8A, in accordance with aspects of the present disclosure;

FIG. 20 is a schematic diagram showing inactivation of a transposome, in accordance with aspects of the present disclosure;

FIG. 21A shows a graph illustrating a distribution of fragment sizes before being subject to zSLTCs, in accordance with aspects of the present disclosure;

FIG. 21B shows a graph illustrating a distribution of fragment sizes bound to inactive surface-linked transposome complexes (zSLTCs) having a first density of inactive transposomes bound to the zSLTCs, in accordance with aspects of the present disclosure;

FIG. 21C shows a graph illustrating a distribution of fragment sizes bound to zSLTCs having a second density of inactive transposomes bound to the zSLTCs, in accordance with aspects of the present disclosure;

FIG. 21D shows a graph illustrating a distribution of fragment sizes bound to zSLTCs having a third density inactive transposomes bound to the zSLTCs T, in accordance with aspects of the present disclosure;

FIG. 22A shows gene expression analysis results using a first amount of universal human reference (UHR) RNA, in accordance with aspects of the present disclosure;

FIG. 22B shows a gene expression analysis results using a second amount of UHR RNA, in accordance with aspects of the present disclosure;

FIG. 23A shows a gene expression analysis results using a first amount of human brain reference (HBR) RNA, in accordance with aspects of the present disclosure;

FIG. 23B shows a gene expression analysis results using a second amount of HBR RNA, in accordance with aspects of the present disclosure;

FIG. 24A shows a distribution of nucleic acids obtained using normalization techniques without zBLTs, in accordance with aspects of the present disclosure;

FIG. 24B shows a distribution of nucleic acids obtained using normalization techniques with zBLTs, in accordance with aspects of the present disclosure;

FIG. 25A shows a distribution of different normalized genes obtained with manual normalization; and

FIG. 25B shows a distribution of different normalized genes obtained with zBLTs.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

Library preparation for downstream processing and analysis, such as for nucleic acid sequencing, generally involves fragmenting a nucleic acid (e.g. genomic DNA) to generate fragments (e.g., nucleic acid fragments) that are subsequently amplified and sequenced. Depending on the fragment preparation technique, the generated fragments may have a relatively broad size range, such as between 10 base pairs to 1000s base pairs. At least in some instances, the instruments that perform the sequencing of the generated fragments may only operate on fragments within a particular fragment size range, and, as such, not all of the fragments may be capable of being sequenced by the instrument. Thus, fragments outside of an operable size range are not used to generate sequencing data and are wasted. For low concentration samples, this waste may result in low sequencing coverage and a reduction of sequencing data quality.

Certain techniques such as using an electrophoretic gel, using coated magnetic beads that can be reformulated to enable size-selection, and the like, may be utilized to select nucleic acid fragments having the particular fragment size range appropriate for the instrument. However, such techniques may nonetheless result in a discarding of a significant portion of the nucleic acid sample consisting of fragments that are not within the particular size range that is appropriate for the instrument. Certain techniques, such as bead-linked transposome methods of Nextera Flex and Nextera Flex for Enrichment impart a greater control over the quantity and reproducibility of the fragment sizes generated. However, the distribution of fragment sizes may still be relatively broad for certain applications and may involve additional size-selection to be done, which may result in discarding over- and under-sized fragments. Additionally, separating (e.g., during size selection) the relatively broad fragment size may be time consuming. Accordingly, it is beneficial to generate fragments of nucleic acids in a size-controlled manner or having relatively narrow size ranges while also limiting an amount of the fragments of nucleic acids not within the particular size range that are discarded.

Accordingly, aspects of the present disclosure relate to methods, compositions, and kits, and, in particular, methods, compositions, and kits for fragmenting nucleic acid to generate fragments having a particular size or size range. Certain techniques for fragmenting a nucleic acid include tagmenting or performing a tagmentation reaction using a transposome.

A transposome is a protein-DNA complex that includes a transposase (e.g., Tn5 enzyme) and a transposon. The transposome is capable of tagmenting a target nucleic acid sample via a transposition reaction. In general, “tagmenting”, or performing a “tagmentation reaction”, involves the transposon end sequence joining to the nucleic acid, thereby tagging (i.e., the transferred strand joining) the nucleic acid, and simultaneously cleaving the nucleic acid to produce fragments. The transposome inserts as a dimer, as discussed in further detail herein, such that the transposome tagments (e.g., tags and fragments) both strands of the nucleic acid. More specifically, two transposase enzymes in the transposome dimer (i.e., each transposome having one of the two transposases) insert into a different strand of a double-stranded nucleic acid. Each transposase enzyme of the transposome dimer nicks its respective nucleic strand and ligates the transferred strand of a transposome (e.g., of the transposome dimer) to the nicked end of the nucleic acid. The non-transferred strand of the transposome may be hybridized to the transferred strand, but is not ligated by the transposase enzyme. Tagmenting a target nucleic acid using multiple transposomes (i.e., with each transposome being a dimer) involves the transposon end sequences of each of the transposomes joining to a different location along the target nucleic acid and cleaving the target nucleic acid at the different locations. As such, a target fragment forms between two neighboring locations along a respective strand (e.g., two locations having no intervening transposome) where the transposon end sequences of two transposomes joined, and the target fragment has a length that correspond to a distance between the two neighboring locations. Furthermore, the target fragment is tagmented, and thus include two transposon end sequences at opposing terminal ends of the target fragment. That is, the target fragment includes a first transposon end sequence at a first terminal end originating from a first transposome of the two transposomes. Additionally, the tagmented target fragment includes a second transposon end sequence at a second terminal end (e.g., different from the first terminal end) originating from a second transposome of the two transposomes. It should be noted that although the above disclosure describes both of the transposase enzymes of a dimer tagmenting a nucleic, it should be noted that, at least in some instances, only one of the transposase enzymes may be tagment (i.e., one of the transposase enzymes may be inactivated, as described in further detail herein).

At least in some instances, the terminal ends of the target fragments are single-stranded along a portion of the target fragment after being tagmented (e.g., having a single-stranded gap). For example, the target fragment may include a single-stranded gap extending along a portion of the target fragment adjacent to a transposon end sequence (e.g., at the first terminal end and/or the second terminal end). It should be noted that a gap fill reaction may be performed to add additional nucleic acids along the single-stranded gap such that the target nucleic acid is double stranded along the portion of the target fragment adjacent to the transposon end sequence.

As discussed in more detail herein, the disclosed techniques include using a transposome complex (e.g., a concatenated complex) formed of multiple enzymes (e.g., transposomes) that may each bind onto a region of a target nucleic acid. As discussed in more detail herein, the transposome complex may include combinations of active transposomes and inactive transposomes. In some embodiments, the transposome complex may include inactive transposomes that are each coupled to one another and a first active transposome coupled to a first end of the transposome complex and a second active transposome coupled to a second end of the transposome complex. In general, the active transposomes are catalytically active (e.g., the transposomes are not inactivated due to chemical modifications or heat), and thus are capable of inserting into a target nucleic acid. An “inactive” transposome refers to a catalytically inactive transposome that is unable to join (e.g., insert via ligation) into a target nucleic acid and/or prevent or remove the ability of the transposase to nick a nucleic acid strand. In certain embodiments, the transposome may be inactivated via a selective mutation to remove or reduce enzymatic activity. At least in some instances, the inactive transposome may still bind to the target nucleic acid. At least in some instances, the inactive transposome may be inactive due to inactivation of the transposase of the transposome, such as by modifying an amino acid sequence of the transposase. In some embodiments, the transposome may be in inactive due to modifications of oligonucleotides forming adaptors of the transposome that render the transposome inactive, while the transposase may still be active. As such, when each transposome of the transposome complex (e.g. the inactive transposomes, the first active transposome, and the second active transposome) bind to the target nucleic acid respective regions, the target nucleic acid may only be fragmented at the regions where the first active transposome the second active transposome are bound thereby generating a fragment having a length that is proportional to the length of the transposome complex or the footprint of the bound transposome complex on the target nucleic acid. Thus, a tagmentation reaction using the disclosed transposome complex may generate multiple fragments each having approximately the same length. Moreover, by tuning (e.g., increasing or decreasing) the number of inactive transposomes, the lengths of the fragments generated via tagmentation using the disclosed transposome complex may be controlled. As such, the disclosed techniques may reduce the amount of a nucleic acid that goes unused, which may be beneficial to applications where an amount of the nucleic acid is limited. Further, the disclosed techniques may improve the speed at which the fragments are generated by reducing a number of additional steps to be performed on the fragments, such as size-selection.

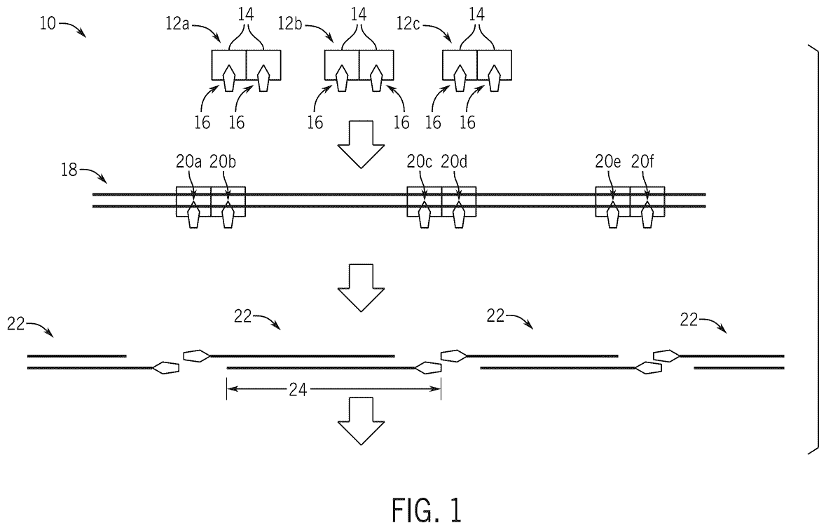

With the foregoing in mind, FIG. 1 shows a schematic flow diagram 10 illustrating the transposase-catalyzed insertion of transposome end sequences into a nucleic acid to generate fragments of the nucleic acid that may be performed in conjunction with the size-controlled fragment generation techniques as provided herein. In the illustrated embodiment, multiple transposomes 12 including at least one transposase 14 and a transposon end sequence 16 are provided to a target nucleic acid 18. In general, the transposon end sequence 16 may be part of a transposome complex, or a transposome composition that is capable of inserting or transposing the transposon end sequence 16 into a target nucleic acid, such when the transposase 14 is incubated with the target nucleic acid 18 in an in vitro transposition reaction. In general, the transposase 14 (e.g., an integrase or integration enzyme) recognizes and binds to the transposon end sequence 16 to form the transposome 12. For example, the transposon end sequence 16 may be a nucleic acid capable of forming a complex with a transposase 14 such as a hyperactive Tn5 transposase. In this example, the transposon end sequence 16 generally includes a transferred transposon end sequence (e.g., a transferred strand) and a non-transferred transposon end sequence (e.g., non-transferred strand). The 3′-end of the transferred strand is joined or transferred to the target nucleic acid 18 in an in vitro transposition reaction. The non-transferred strand, which exhibits a transposon end sequence that is complementary to the transferred transposon end sequence, is not joined or transferred to the target DNA in an in vitro transposition reaction.

Other examples of transposon end sequences 16 include but are not limited to wild-type, derivative or mutant transposon end sequences that form a complex with a transposase 14 chosen from among a wild-type, derivative or mutant form of the transposase. For example, the transposon end sequence may be a wild-type or mutant form of Tn5 transposase and MuA transposase. In some embodiments, the transposon end sequence 16 that binds to the transposase 14 are of a suitable size to provide selectivity of the binding between the transposon end sequence 16 and the transposase 14. For example, the transposon end sequences of the Tn5-derived EZ-Tn5™ transposon end sequences comprise only 19 nucleotides, whereas some other transposases require much larger end sequences for transposition (e.g., MuA transposase utilizing transposon end sequences of approximately 51 nucleotides).

In some embodiments, one or more additional nucleotide sequences may be attached to 5′-end of the transferred strand or 3′-end of the non-transferred strand. For example, the one or more additional nucleotide sequences may include barcodes, universal molecular identifiers (UMIs), or other adaptor sequences, that may facilitate sequencing of the target nucleic acid 18 by enabling identification of a relative ordering of the fragments.

Referring back to FIG. 1, the transposon end sequence 16 of each transposome 12 joins to the target nucleic acid 18 at a respective region 20. In the depicted embodiment, three transposomes 12 are shown: a first transposome 12a, a second transposome 12b, and a third transposome 12c. The transposon end sequences 16 of the first transposome 12a join to the strands of the target nucleic acid 18 at regions 20a and 20b. The transposon end sequences 16 of the second transposome 12b join to the strands of the target nucleic acid 18 at regions 20c and 20d. The transposon end sequences 16 of the third transposome 12c join to the strands of the target nucleic acid 18 at regions 20e and 20f. Accordingly, when the transposome 12 fragments the target nucleic acid 18 (e.g., using the transposase 14), the transposome 12 generates target fragments 22 (e.g., nucleic acid fragments), which each have a length 24 (i.e., length 24 of the fragments 22 is shown) that is proportional the distance between two regions 20 where the two flanking transposon end sequences 16 join the target nucleic acid 18 and representative of a nucleotide base or base pair length of the fragment 22. For example, the length 24 shown in the illustrated embodiment may be proportional to a length between the region 20a and the region 20c. It should be noted that the transposon end sequences 16 may be double-stranded and joining of the transposon end sequences 16 to the strands of the target nucleic acid 18 may generate target fragments 22 that each include a single stranded gap (e.g., approximately 9 base pairs) that extends along a terminal end of the target fragments 18 to the transposon end sequences.

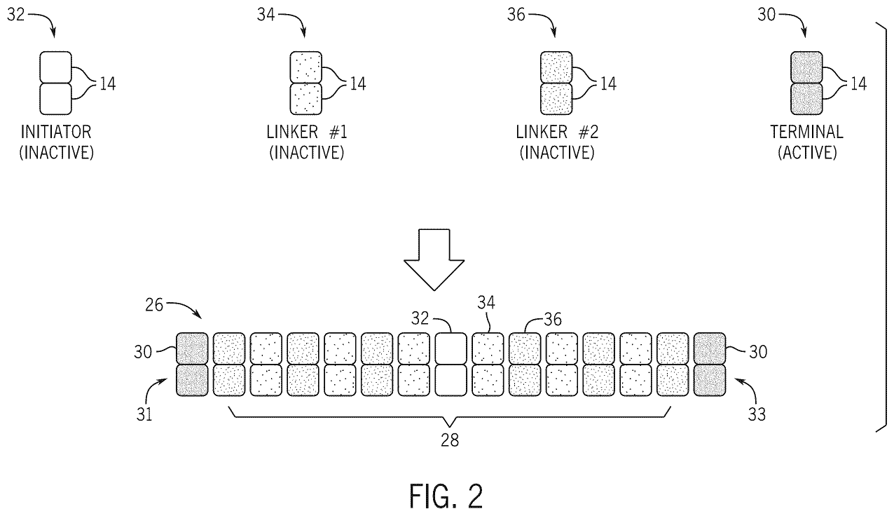

The sizes (e.g., the length 24) of the fragments 22 generated by the transposome 12 may have a relatively large size distribution, and thus, at least a portion of the fragments 22 may be discarded due to being too large or too small for certain applications, such as for sequencing by a particular instrument. To generate fragments having a controllable size distribution, a transposome complex formed from multiple inactive enzymes (e.g., transposases 14) and multiple active transposomes may be utilized to fragment the target nucleic acid. To illustrate this, FIG. 2 shows a schematic diagram of a transposome complex 26 that may provide size-controlled strands of DNA for sequencing.

In the illustrated embodiment, the individual transposome complex 26 includes multiple inactive transposomes 28 and active transposomes 30 each having associated transposases 14. As shown, the inactive transposome 28 includes 13 inactive transposomes 28. However, the transposome complex 26 may have any suitable number of inactive transposomes 28. In one example, the transposome complex 26 as provided herein includes a first active transposome 30 separated from a second active transposome 30 by one or more inactive transposomes 28. The active transposomes 30 and the one or more inactive transposomes 28 are coupled to (e.g., linked to, bound to, hybridized to via complementary sequences) one another. In one embodiment, the intervening inactive transposome 28 or inactive transposomes 28 (positioned between the first active transposome 30 and the second active transposome 30) are linked to neighboring transposomes that may be active or inactive, depending on the particular arrangement of the transposome complex 26. The active transposomes 30 form ends (a first end 31, a second end 33) of the transposome complex 26 such that each active transposome 30 at the ends 31, 33 is only linked to one neighboring transposome (e.g., an inactive transposome 28). In an embodiment, there is a single intervening inactive transposome 28 that is linked to both terminal active transposomes 30 at the ends 31, 33 of the transposome complex. In an embodiment, the ratio of active transposomes 30 to inactive transposomes 28 in the transposome complex 26 is 2:1, 2:2, 2:3, 2:4, 2:5, 2:6, 2:7, 2:8, 2:9, 2:10, 2:15, 2:20, 2:25, 2:30, 2:40, or 2:N.

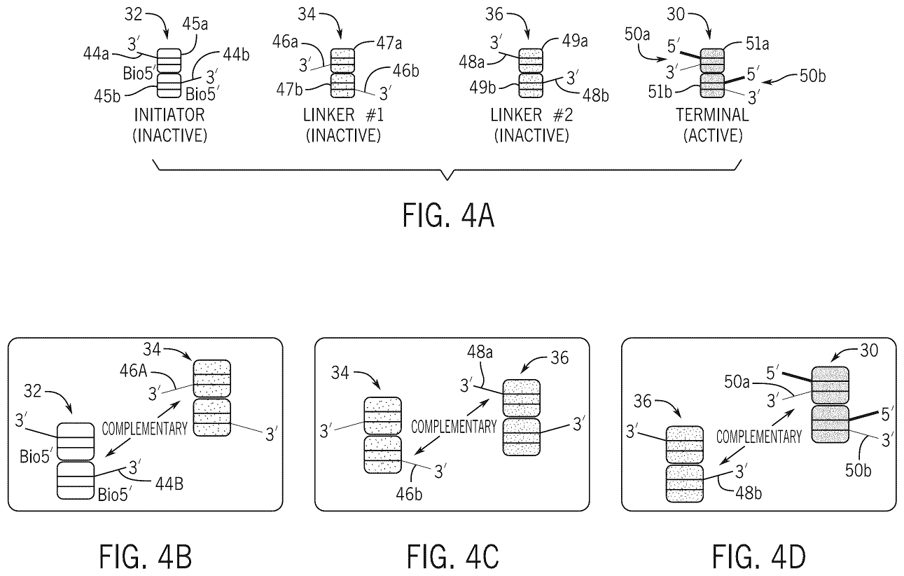

However, as discussed herein, the number, arrangement, and/or type of the intervening inactive transposomes 28 between the terminal active transposomes 30 may be selected to provide desired length control or to facilitate particular sequencing techniques. In the depicted embodiment, the transposome complex 26 includes inactive transposomes 28. For example, the depicted embodiment includes a first inactive transposome, a second inactive transposome, and a third inactive transposome, etc. In an embodiment, each of the inactive transposomes 28 within the transposome complex 26 may differ structurally from adjacent or neighboring transposomes due to a different end sequence and/or linking sequence that couples the inactive transposomes 28 together, i.e., to neighboring transposomes. It should be noted that providing each of the inactive transposomes 28 with a different adaptor may enable the transposome complex 26 to grow in a controlled manner. That is, each adaptor for each of the inactive transposomes may provide selectivity of neighbor binding, as discussed in more detail with respect to FIG. 4. Thus, as illustrated, the inactive transposomes 28 may include an initiator transposome 32 that forms a seed from which the transposome complex 26 is grown and various linker transposomes 34, 36 that may include sequences that are complementary to one another and/or the initiator transposome 32.

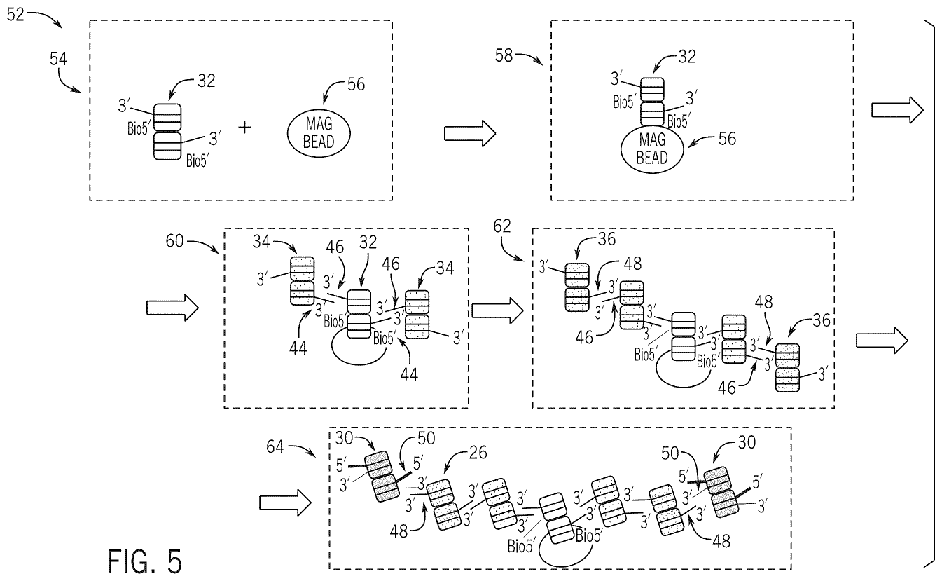

In some embodiments, the adaptor may be an adaptor sequence (e.g., oligonucleotide adaptor) that is specific to each type of inactive transposome. For example, the initiator transposome 32 may include a first adaptor (e.g., a first adaptor sequence) that has a nucleic acid sequence that is configured to hybridize to (via complementary binding) a second adaptor (e.g., a second adaptor sequence) of the linker transposome 34. Additionally, the linker transposome 36 may include a third adaptor (e.g. a third adaptor sequence) that is configured to hybridize to the second adaptor but not the first adaptor. As such, when the inactive transposomes 28 are being assembled, such as by adding each of the inactive transposomes sequentially in solution, which is discussed in more detail with respect to FIG. 5, the growth, and thus the length of the transposome complex 26, can be controlled through sequential addition of each the different types of inactive transposomes 28.

For example, the transposome complex 26 may include any number of inactive transposomes 28 such that the resulting length of the fragment generated using the transposome complex 26 is greater than 50 base pairs, 100 base pairs, 500 base pairs, or greater than 1000 base pairs. The generated nucleic acid fragment lengths may be between 50-150 base pairs, 50-500 base pairs, 150-500 base pairs, 500-1000 base pairs. As discussed above, the active transposomes 30 are capable of inserting into a target nucleic acid, and, thus, are catalytically active. For example, the active transposomes 30 may have catalytically active ends that may insert a sequence into a nucleic acid. As also discussed herein, an “inactive” transposome, such as inactive transposome 28, refers to a catalytically inactive transposome (e.g., due to modification of an adaptor of the transposome 28 or the transposase) that is unable to join (e.g., insert) into a target nucleic acid (e.g., the target nucleic acid 18), but the inactive transposome 28 nonetheless still binds to the target nucleic acid 18.

In general, the inactive transposome 28 may be deactivated using suitable chemical or heat inactivation techniques, such as via chemical modifications or by blocking an end sequence of the transposase of the transposome. For example, such techniques for deactivating a transposome to generate an inactive transposome 28 include, but are not limited to, heating the transposase, dephosphorylating 5′-end of the transposase, and blocking 3′-end with a chemical modification. While the active transposomes 30 and the inactive transposomes 28 are described as being different (i.e., active or inactive), it should be noted that in some embodiments, the active transposomes 30 and the inactive transposomes 28 may include the same type of integrase (e.g., transposase) enzyme.

Accordingly, the transposome complex 26, incubated with a target nucleic acid 18, would join to target nucleic acid 18 using the active transposomes while the inactive transposomes each bind to a respective portion of the target nucleic acid 18. That is, 3′-end of the transposon ends of the active transposomes 30 would join to 5′-end of the target nucleic acid 18. It should be noted that controlling the number of inactive transposomes 28 may be used to control the length of the DNA strand that is ultimately fragmented, as discussed in more detail with respect to FIG. 5.

In the illustrated embodiment, the transposomes (e.g., the active transposomes 30 and the inactive transposomes 28) are dimers. That is, each transposome includes a dimer, monomers of the dimer having a transposase enzyme (e.g., Tn5 transposase) coupled to a transposon or other adaptor sequence. For example, the active transposome 30 includes an active transposome dimer and the inactive transposomes 28 includes inactive transposome dimers. In some embodiments, a portion of the transposomes (e.g., the holo-transposome) may be homodimers. In some embodiments, the transposomes may be linked dimers. That is, the monomers of the dimer may be linked, such as by a posttranslational addition of a linker or the protein of the transposome may be expressed as a fusion in tandem when manufactured. For example, the transposome may be a gene fusion of Tn5 transposase resulting in a single transposase protein backbone having two identical domains (e.g., both being a Tn5 transposase). In some embodiments, a portion of the transposomes may be heterodimers. It should be noted that the transposomes may generally include other types of integration capable of binding to DNA. For example, DNA binding enzymes may include, but are not limited to, a Crispr/Cas protein.

Accordingly, the transposome complex 26 may be used to bind to DNA and generate size-controlled fragments. It should be noted that each of the enzymes of the transposome complex 26 may be capable of binding to a nucleic acid, irrespective of whether or not the enzymes are catalytically active. That is, the inactive transposomes 28 may still permit binding to the target nucleic acid 18, although the transposases of the inactive transposomes 28 are catalytically inactive. At least in some instances, an initial binding of one or more enzymes in the complex to a nucleic acid may elicit a cooperative effect, binding the remaining enzymes of the complex 26 to the same nucleic acid molecule. The result is that the transposome complex 26 may position the active transposomes 30 (e.g., active terminal transposomes) of the complex in ‘cis’ on the same DNA molecule. It should be noted that because each of the transposomes of the transposome complex 26 is capable of binding to the nucleic acid, the transposome complex 26 may bind to the nucleic acid cooperatively and in ‘cis.’ By binding in ‘cis’, the size of the fragmented nucleic acid may be proportional to the number of transposomes of the transposome complex. That is, these active transposomes 30 subsequently cleave the DNA at a fixed distance (e.g., size) dictated by the length or spatial separation distance between the pair of active terminal transposomes 30 in the transposome complex 26. Moreover, tuning the ratio of the transposome complexes 26 to the nucleic acid (e.g., DNA) substrate or target nucleic acid 18 of the sample of interest may facilitate the fragmenting of the nucleic acid to a uniform size. For example, the ratio of the transposome complex 26 to the nucleic acid where the transposome complex 26 is in excess may facilitate fragmenting the nucleic acid to a uniform size. Additionally, the amount of uncovered or unused nucleic acid may be reduced by increasing the ratio of the transposome complex 26, thereby minimizing or reducing the amount of the nucleic acid that is unused or discarded. In another embodiment, the ratio of the nucleic acid to the transposome complex 26 is in excess, which may generate two populations of fragments having different size distributions. For example, a first population of fragments corresponding to the region of the nucleic acid bound by the transposome complex 26 may have a uniform size distribution, and a second population of the fragments corresponding to the unbound region of the nucleic acid may have a random size distribution. As the second population is not bound by the transposome complex 26, the second population may be digested, such as by a nuclease that cleaves accessible double-stranded nucleic acids.

At least in some instances, the transposome complex 26 binding the target nucleic acid and the active transposomes 30 of the transposome complex 26 cleaving the target nucleic acid (e.g., a cleavage step) may be separated by a time duration. For example, the active transposomes 30 may be activated to cleave the target nucleic acid. That is, the transposome complex 26 may be provided to the target nucleic acid, and after the time duration, which may correspond to a period where the transposomes of the transposome complex 26 are binding to the nucleic acid, the active transposomes may be activated, such as by providing a salt (e.g., a magnesium containing salt) to or increasing a temperature of a solution including the transposome complex 26 and the target nucleic acid. Providing a time duration between the binding and the cleaving of the target nucleic acid may increase the likelihood of the transposomes of the transposome complex 26 binding cooperatively.

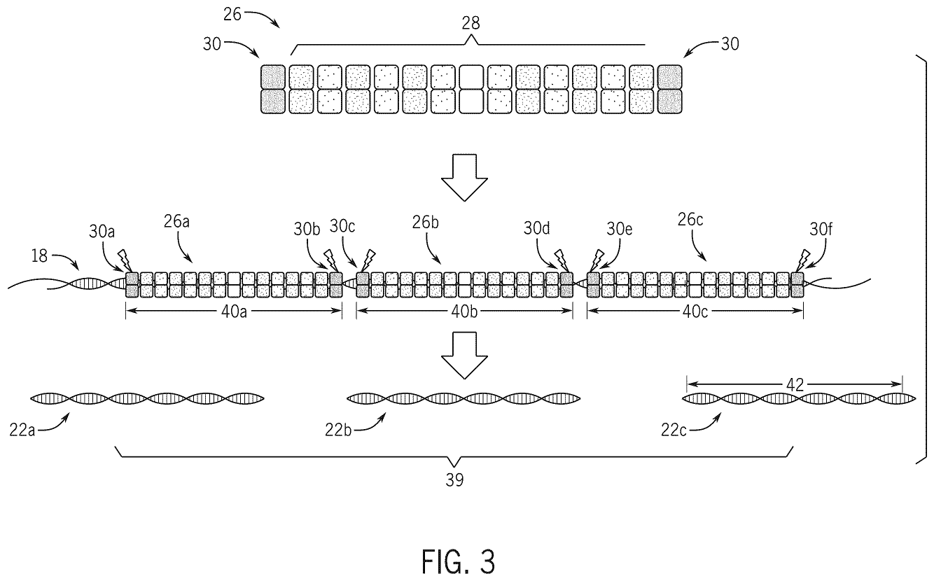

FIG. 3 shows a flow diagram for preparing a nucleic acid library using transposome complexes 26 that bind to a target nucleic acid 18 and generates fragments 22 with size-controlled lengths. As shown in the illustrated embodiment, the transposome complexes 26 bind a nucleic acid 18 along a length 40 of the nucleic acid 18. It should be noted that, although the transposome complex 26 includes inactive transposomes 28, at least a portion of, or all of the transposomes of the complex 26 bind to the nucleic acid 18. As discussed herein, both the active transposomes 30 and the inactive transposomes 28 bind the nucleic acid 18, irrespective of whether or not the transposomes (e.g., the active transposomes 30 or the inactive transposomes 28) are active. Moreover, an initial binding of one or more transposomes in the transposome complex 26 to the nucleic acid 18 may elicit a cooperative binding effect, which results in each of the remaining unbound transposomes binding to the same nucleic acid 18.

Accordingly, the individual transposome complexes 26 each bind along a respective length 40 of the nucleic acid. That is, the first transposome complex 26a binds along a first length 40a of the target nucleic acid 18, the transposome complex 26b binds along a second length 40b of the target nucleic acid 18, and the third transposome complex 26c binds along a third length 40c of the target nucleic acid 18. Binding to the nucleic acid 18 results in the transposome complex 26 mediating a tagmentation reaction of the nucleic acid 18. As discussed herein, “tagmenting”, or performing a “tagmentation reaction”, involves the transposon end sequence joining to the nucleic acid 18 at the binding site, thereby tagging (i.e., the transferred strand joining) the nucleic acid 18, and simultaneously cleaving the nucleic acid 18 to produce fragments 22 that together may form a nucleic acid library 39. For example, after the transposome end sequences of each active transposome 30 joins onto the target nucleic acid 18, fragments 22 are generated. The fragment 22a forms when 3′-end of the transposon end sequences of the active transposome 30a and 30b joins to the nucleic acid 18 along the length 40a. The fragment 22b forms when 3′-end of the transposon end sequences of the active transposomes 30c and 30d join to the nucleic acid 18 along the length 40b. The fragment 22c forms when 3′-end of the transposon end sequences of the active transposomes 30e and 30f join to the nucleic acid 18 along the length 40c. As the inactive transposomes between the active transposomes 30 do not join to the target nucleic acid 18, the length 42 of the fragment 22 is a function of the binding length encompassed by the transposome complexes 26. Accordingly, the length 42 of the fragments 22 is based upon the number of transposomes (e.g., the active transposomes 26 and the inactive transposomes 28) of the transposome complex 26.

In an embodiment where multiple transposome complexes 26 are provided, each transposome complex 26 has approximately the same length 40 (i.e., the same number of total transposomes or a same transposome arrangement and/or number inactive transposomes 28 and active transposomes 30) relative to one another such that each resulting fragment will have approximately the same length 40. In an embodiment where multiple transposome complexes 26 of different sizes are used, the resulting cleaved nucleic acid fragments 22 will have corresponding different lengths 40.

At least in some instances, a portion of the nucleic acid 18 may be uncovered or not be bound to the transposome complex 26, and thus may not be of a suitable length (e.g., for measurements by an instrument). As such, the uncovered portion of the nucleic acid 18 may be dissolved or digested by suitable means known to one of ordinary skill in the art. In this way, extraneous nucleic acid 18 may be substantially removed from the solution or substrate where the library preparation is occurring. The digesting may occur in conjunction with transposome complex binding such that only the uncovered portion is digested and the covered portion of the nucleic acid 18 is protected by the presence of associated transposome complexes 26. Alternatively, size exclusion methods may be used to filter out the uncovered portions having a first size from the covered portions having a second size.

As discussed above, each of the transposomes (i.e., the inactive transposomes 28 and the active transposomes 30) may capable of only binding to a specific type of the one or more transposomes. To illustrate this, FIGS. 4A-4D (e.g., FIGS. 4A, 4B, 4C, and 4D) show inactive transposomes and active transposomes that each include adaptor sequences that provide selective binding to the inactive transposomes and the active transposomes.

The depicted embodiment of FIG. 4A shows the first inactive transposome 32, the second inactive transposome 34, and the third inactive transposome 36. Each of the transposomes (e.g., the first inactive transposome 32, the second inactive transposome 34, the third inactive transposome 36, and the active transposome 30) each include a respective pair of adaptors. As discussed herein, the transposome may be dimer complexes, and as such, each transposome includes two adaptors. As shown, the first inactive transposome 32 includes a first adaptor 44a and a second adaptor 44b, the second inactive transposome 34 includes a first adaptor 46a and a second adaptor 46b, the third inactive transposome 36 includes a first adaptor 48a and a second adaptor 48b, and the active transposome 30 includes a first adaptor 50a and a second adaptor 50b.

In general, the adaptors 44, 46, 48, and 50 may be an at least partially double-stranded oligonucleotide. In the illustrated embodiment, the adaptors 44, 46, 48, and 50 include a single-stranded overhang on 3′-end. However, in some embodiments, the adaptors 44, 46, 48, and 50 may include a single-stranded overhand on 5′-end. In some embodiments, the adaptors 44, 46, 48, and 50 may be coupled to the respective transposome monomer. For example, the first adaptor 44a may be coupled to a first transposase 45a of the first inactive transposome 32 via a first monomer, and the second adaptor 44b may be coupled to a second transposase 45b of the second inactive transposome 32 via a second monomer. Similarly, the first adaptor 46a may be coupled to a first transposase 47a of the inactive transposome 34 via a first monomer, and the second adaptor 46b may be coupled to a second transposase 47b of the second inactive transposome 34 via a second monomer. The first adaptor 48a may be coupled to a first transposase 49a of the inactive transposome 36 via a first monomer, and the second adaptor 48b may be coupled to a second transposase 49b of the second inactive transposome 36 via a second monomer. In some embodiments, the adaptors 44, 46, 48, 50 may be different for a respective transposome. The adaptors 44a and the 44b may include the same nucleotide sequence as part of a homodimer.

In the depicted embodiment, the active transposome 30 includes a double-stranded adaptor on each active transposase 51 of the transposome dimer. It should be noted that, the first adaptor 50a (e.g., a first oligonucleotide adaptor) of the first active transposome 30 and a second adaptor 50b (e.g., a second oligonucleotide adaptor) of the second active transposome 30 may each comprise a double-stranded transposon end sequence and a single-stranded adaptor sequence on each monomer of respective transposome dimers.

As a non-limiting example of how the adaptors oligonucleotide sequences may be used to form the transposome complex (e.g., the concatenated complex), the Tn5 transposase adaptors are double-stranded oligonucleotides of a fixed sequence known as the Mosaic End (ME) sequence. The strand that is ligated to the target nucleic acid (e.g., target substrate DNA) during tagmentation is referred to as the ‘transfer strand’. It should be noted that 3′OH-end of this strand is transferred and ligated to the target nucleic acid during tagmentation. The complementary strand in a Tn transposome may be referred to as the “non-transfer strand”. In an active transposome enzyme, 5′OH-end is phosphorylated; phosphorylation is necessary for the transposome to be active. The absence of this phosphate renders the transposome catalytically inactive but still capable of binding substrate DNA. The ME duplex may be approximately 19 bp long. For example, the ME duplex may be short at one or both of 5′ end of the transfer strand of the ME or 3′ end of the non-transfer strand of the ME. Additional sequences may be appended to 5′-end of the transfer strand and 3′-end of the non-transfer strand. These additional bases can be of any length and sequence.

In one specific embodiment, the first inactive transposome 32 (e.g., an initiator transposome) comprises a non-transfer strand that has additional sequences appended to its 3′-end. These additional sequences may be complementary to additional sequences appended to the 3′-end of the non-transfer strand of the second inactive transposome 34 (e.g., a first linking transposome), as shown in FIG. 4B. Accordingly, at least a portion (e.g., at least 5 bases, at least 10 bases) of the adaptor sequences 44, 46 are complementary. Additionally, the additional sequences may be the same sequence and polarity to the additional sequences appended to the 3′-end of the non-transfer strand of the third inactive transposome 36 (e.g., a second linking transposome). By definition the additional sequences appended to 3′-end of the non-transfer strand of the second inactive transposome 34 are complementary to the additional sequences appended to 3′-end of the non-transfer strand of the third inactive transposome 36, as shown in FIG. 4C. Moreover the additional sequences appended to 3′-end of the non-transfer strand of the first inactive transposome 32, and being the same sequences as the additional sequences appended to 3′-end of the non-transfer strand of the third inactive transposome 36, are complementary to additional sequences appended to 3′-end of the non-transfer strand of the active transposome 30 (e.g., the terminal transposome), as shown in FIG. 4D. It should be noted that although FIGS. 4A-4D illustrate assembly of the transposome complex 26 via hybridization of 3′-ends of the non-transfer strands of the transposomes, assembly may also be achieved via hybridization of 5′-ends of the non-transfer strands of the transposomes.

Each of the transposomes (e.g., the active transposome 30, the first inactive transposome 32, the second inactive transposome 34, and the third inactive transposome 36) can have additional sequences appended to 5′-end of the transfer strand of these transposomes. The active transposome 30, in particular, may have additional sequences appended to 5′-end of the transfer strand that perform a role later in the preparation of a library such as appending additional functionality, for example, sequences utilized for amplification or attachment of the library to a sequencing flow cell. Such sequences may include universal adaptor sequences, sequencing primers, capture sequences, etc. In one embodiment, 5′-end of the non-transfer strand of the active transposome 30 is phosphorylated. Any of the transposomes may contain a moiety for attachment of the transposome to a surface. For example, 5′-end of the transfer strand of the first inactive transposome may be biotinylated such that it binds to streptavidin coated magnetic bead. In some embodiments, additional sequences may be appended to 3′-end of the non-transferred strand. For example, 3′-end of the non-transferred strand may include a sequence capable of being recognized and bound to by certain enzymes, such as a polymerase used in a gap-filling reaction. As such, after tagmentation has occurred from the active transposome 30 and the transfer strand is ligated to the DNA substrate, the non-transferred strand can also be ligated to the DNA substrate, such as by using a non-strand displacing polymerase and a ligase. It should be noted that the transfer strand or the non-transfer strand may include the additional sequences, which may facilitate the addition of further adaptor sequences (e.g., by primer extension, ligation).

FIG. 5 is a flow diagram of a method 52 for preparing a transposome complex 26. At block 54, the first inactive transposome 32 (e.g., an initiator transposome) is provided. In some embodiments, providing the transposome complex may include providing a substrate 56, shown here as a magnetic bead. At block 58, the first inactive transposome 32 is attached to the substrate 56 (e.g., via 5′ end (i.e., “Bio5′”) of the first inactive transposome 32). For example, the initiator transposome 32 is attached to a streptavidin magnetic bead. In other embodiments, the transposome complex 26 is prepared in solution. In an embodiment where a support surface substrate 56 is used, the substrate 56 may be washed to remove any of the first inactive transposome 32 that remains in solution or did not bind to the substrate.

At block 60, one or more of the second inactive transposome 34 (e.g., a linking transposome) is added and hybridized to the initiator transposome via its complementary sequences and then washed to remove unbound transposome. As discussed with respect to FIGS. 4A-4D, the second inactive transposome 34 may include adaptors 46 that are complementary to the adaptors 44 of the first inactive transposome 32. As such, the adaptors 44 of the first inactive transposome 32 may couple, bind, or hybridize to the adaptors 46 of the second inactive transposome 34. In such embodiments where both adaptors 44 of the first inactive transposome 32 are complementary to both adaptors 46 of the second inactive transposome 34, the second inactive transposome 34 may bind to both sides (e.g., both adaptors 44) of the first inactive transposome 32. In any case, once one or more of the second inactive transposomes 34 have hybridized to one or more of the first inactive transposome 32, the substrate 56 may be washed to remove any second inactive transposomes 34 remaining in solution (i.e., are not bound to the first inactive transposome 34). Additionally or alternatively, the second inactive transposome 34 and the first inactive transposome may be crosslinked. At least in some instances, crosslinking the second inactive transposome 34 and the first inactive transposome together may improve the rigidity or robustness of the transposome complex 26. Further, crosslinking may improve the size-control of the transposome complex 26. At least in some instances, crosslinking may improve the stability of the transposome complex 26 by preventing or substantially reducing monomeric exchange between transposomes of the transposome complex 26. In some embodiments, the transposomes of the transposome complex 26 may include stabilizers, such as a locked nucleic acids (LNA), which may provide additional stability to the transposomes of the transposome complex 26.

At block 62, the third inactive transposome 36 (e.g., a second linking transposome) is added and hybridized to the second transposome (e.g., the second inactive transposome 34) via its complementary sequences, in a generally similar manner as described with respect to the hybridization of the second inactive transposome 34 to the first inactive transposome 32. In some embodiments, blocks 60 and 62 can be repeated multiple times to add additional inactive transposomes (e.g., linker transposomes, the second inactive transposome 34, the third inactive transposome 36) to the transposome complex 26 thereby increasing the length of the transposome, which increases the size of the fragments generated using the transposome complex 26. When the transposome complex 26 reaches a predetermined length, the active transposome 30 (e.g., a terminal transposome), at block 64, may be hybridized to the third inactive transposome 36, thus providing catalytically-active ends to the transposome complex 26 (e.g., concatenated complex). The inactive transposomes 28 of the transposome complex 26 may be provided as already-inactivated individual transposomes or may be bulk-inactivated after being linked together but before addition of the active transposomes 30. It should be noted that by providing different adaptors for each of the first inactive transposomes 32, the second inactive transposomes 34, and the third inactive transposomes 36 may prevent uncontrolled growth of the transposome complex 26. For example, having the adaptors being different may prevent multiple inactive transposomes binding to a particular end of the transposome complex 26 during blocks 60 and 62.

As discussed herein, the disclosed transposome complex may be used to prepare a nucleic acid library, such as a sequencing library, to generate fragments of the DNA having controllable lengths. The DNA is cleaved with the transposome complex 26. For example, the transposon end sequence may include the transferred DNA strand and a non-transferred strand of DNA that may contain a 19 base pairs (bp) mosaic end sequence or truncated DNA sequence. The non-transferred strand (e.g., with or without nuclease protecting and/or chain termination groups, e.g. phosphorothioate and/or dideoxy) then dissociated from the transferred strand and a replacement oligo (which may contain additional DNA sequence such as a sequencing tag) is annealed to the complementary transferred strand sequence with or without nuclease protective groups (e.g. phosphorothioates). Non-displacing nucleic acid modifying enzymes may be used consisting of a DNA polymerase (e.g. thermostable polymerases, or nonthermostable polymerases such as DNA polymerase I or Klenow fragment exo⁻) and a DNA ligase. The DNA polymerases and ligase are utilized to fill in and ligate the gap between the mono-tagged DNA and replacement oligonucleotide resulting in a piece of dsDNA with a covalently attached 5′ and a 3′ tag. Alternatively, an oligonucleotide can be provided to fill in the gap, followed by ligation.

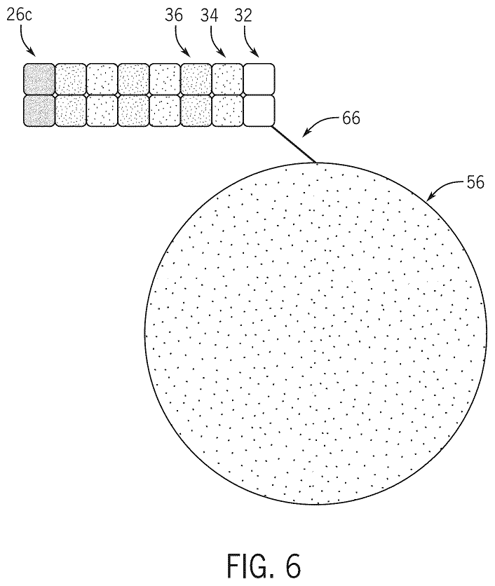

As generally discussed above, the second inactive transposome 34 may hybridize to both sides of the first inactive transposome 32. Thus, the transposome complex 26 may be symmetric in that there are inactive transposomes growing from opposing sides of the first inactive transposome 32. At least in some instances, the transposome complex 26 may grow asymmetrically about the first inactive transposome 32. To illustrate this, FIG. 6 shows a schematic diagram of a transposome complex 26 that has grown asymmetrically. In the depicted embodiment, the first inactive transposome 32 is coupled to the substrate 56 (e.g., a magnetic bead) via a linkage 66. The linkage 66 may be a hybridization between two single stranded nucleic acids that are bound to the first inactive transposome 32 and the substrate 56, respectively. In any case, by binding one side of the first inactive transposome 32 to the substrate 56, the second inactive transposome 34 may only hybridize to the opposing side of the first inactive transposome. While the depicted embodiment shows a transposome complex 26 that has grown asymmetrically, it should be appreciated that a transposome complex 26 may also grow symmetrically about the first inactive transposome 32.

The transposome complexes 26, after formation by the disclosed techniques, may be purified or otherwise undergo selection steps (e.g., molecular weight-based selection) to form a composition enriched for transposome complexes 26 that are likely to be a same size and have a same number of inactive and active transposomes. At least in some instances, the transposome complex 26 may remain bound to the substrate 56 for use in a library preparation reaction. For example, during the library preparation reaction, multiple target nucleic acids may be provided to a solution including multiple transposome complexes 26, and each transposome complex 26 may be bound to a respective substrate 56.

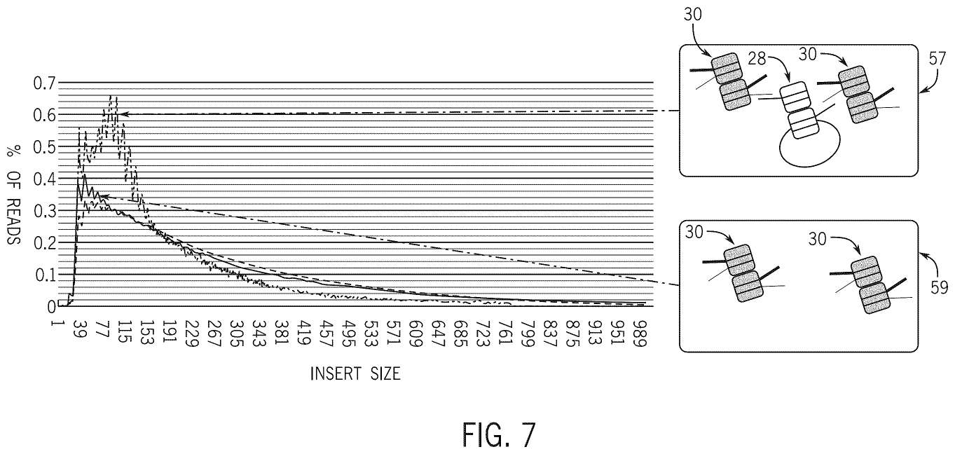

FIG. 7 shows a plot of template insert sizes that result from sequencing DNA libraries, in accordance with aspects of the present disclosure. More specifically, FIG. 7 represents an insert size plot resulting from a paired-end sequencing experiment. The read pairs (i.e., originating from the ends of the template being sequenced) are mapped to the reference genome and used to determine the size of the template insert. This experiment set illustrates a comparison between two embodiments of transposomes. In one case (e.g., the embodiment illustrated in the box 57), a concatenated transposome complex was created comprising a single inactive ‘anchor’ transposome 28 coupled to a bead surface, and with two active transposomes 30 bound via complementary adaptors. In the other embodiment (e.g., illustrated in the box 59), free active transposomes 30 were used. In both cases, an equivalent amount of active transposome was used in a tagmentation experiment. The results indicates that when the transposomes are concatenated, the modal distribution of insert sizes is greater than when not concatenated, illustrating that the disclosure transposome complex having concatenated transposomes can modulate and enlarge insert sizes

Accordingly, aspects of the present disclosure relate to preparing a transposome complex that enables the generation of size-controlled nucleic acids, such as during library preparation. In general, the disclosed transposome complex has multiple inactive transposomes that are each coupled to a neighboring inactive transposome via adaptors (e.g., oligonucleotide adaptor sequences. Additionally, the disclosed transposome complex includes active transposomes that are coupled to the inactive transposomes at the end of the multiple inactive transposomes. As discussed herein, “active” or “inactive” refers to an ability of inability of the transposome, or transposase of the transposome, to tagment a nucleic acid. For example, an active transposome may have an available transferred strand. However, while the disclosed transposome complex may include inactive transposomes, the inactive transposomes may still bind to a target nucleic acid. As such, when the disclosed transposome complex is provided to a nucleic acid, at least a portion of the transposomes (e.g., the active transposomes and the inactive transposomes) may bind to the nucleic acid. After binding to the nucleic acid, the active transposomes may tagment the nucleic, thereby inserting a transposon end sequence into the nucleic acid and fragment the nucleic acid, and thus producing portions of the nucleic acid that were bound to the multiple transposomes. These portions of the nucleic acid that were bound to the transposomes, after fragmentation, are fragments having a size that is approximately equal to the length of the transposome complex. It should be noted that by modifying the number of transposomes (e.g., the inactive transposomes) provided to the transposome complex, the length of the transposome complex, and thus the size of the fragments that are ultimately produced by the transposome complex may be tuned. Accordingly, the disclosed transposome complex may reduce the amount of fragments that are discarded due to being of an inappropriate size of certain instrument by generating such a transposome complex having a number of inactive transposomes inactive transposomes corresponding to a length (e.g. number of base pairs) suitable for the particular size range of an instrument.

As provided herein, a “transposase” may refer to an enzyme that is capable of forming a functional complex with a transposon end-containing composition (e.g., transposons, transposon ends, transposon end compositions) and catalyzing insertion or transposition of the transposon end-containing composition into the double-stranded target DNA with which it is incubated in an in vitro transposition reaction.

The term “transposon end” may refer to DNA that includes the nucleotide sequences (the “transposon end sequences”) that are necessary to form the complex with the transposase or integrase enzyme that is functional in an in vitro transposition reaction. A transposon end forms a “complex” or a “transposome complex” or a “transposome composition” with a transposase or integrase that recognizes and binds to the transposon end, and which complex is capable of inserting or transposing the transposon end into target DNA with which it is incubated in an in vitro transposition reaction. A transposon end exhibits two complementary sequences consisting of a “transferred transposon end sequence” or “transferred strand” and a “non-transferred transposon end sequence,” or “non transferred strand” For example, one transposon end that forms a complex with a hyperactive Tn5 transposase (e.g., EZ-Tn5™ Transposase, EPICENTRE Biotechnologies, Madison, Wis., USA) that is active in an in vitro transposition reaction comprises a transferred strand that exhibits a “transferred transposon end sequence” as follows:

(SEQ ID NO: 1)
5′ AGATGTGTATAAGAGACAG 3′,

and a non-transferred strand that exhibits a “non-transferred transposon end sequence” as follows:

(SEQ ID NO: 2)
5′ CTGTCT CTTATACACATCT 3′.

The 3′-end of a transferred strand in an active transposome (i.e., active transposome 30) is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction. The inactive transposomes 28, as provided herein, may include all or part of the transposon end sequence or a modified transposon end sequence that results in inactivation or that renders the transposase inactive.

In some embodiments, the transferred strand and non-transferred strand are covalently joined. For example, in some embodiments, the transferred and non-transferred strand sequences are provided on a single oligonucleotide, e.g., in a hairpin configuration. As such, although the free end of the non-transferred strand is not joined to the target DNA directly by the transposition reaction, the non-transferred strand becomes attached to the DNA fragment indirectly, because the non-transferred strand is linked to the transferred strand by the loop of the hairpin structure.

A “transposon end composition” means a composition comprising a transposon end (i.e., the minimum double-stranded DNA segment that is capable of acting with a transposase to undergo a transposition reaction), optionally plus additional sequence or sequences. 5′-of the transferred transposon end sequence and/or 3′-of the non-transferred transposon end sequence. For example, a transposon end attached to a tag is a “transposon end composition.” In some embodiments, the transposon end composition comprises or consists of two transposon end oligonucleotides consisting of the “transferred transposon end oligonucleotide” or “transferred strand” and the “non-transferred strand end oligonucleotide,” or “non-transferred strand” which, in combination, exhibit the sequences of the transposon end, and in which one or both strand comprise additional sequence.

However, in some embodiments, the transposon end composition comprises or consists of at least one transposon end oligonucleotide that exhibits one or more other nucleotide sequences in addition to the transposon end sequences. Thus, in some embodiments, the transposon end composition comprises a transferred strand that exhibits one or more other nucleotide sequences 5′-of the transferred transposon end sequence, which one or more other nucleotide sequences are also exhibited by the tag. Thus, in addition to the transferred transposon end sequence, the tag can have one or more other tag portions or tag domains.

As used herein, a “tag portion” or a “tag domain” means a portion or domain of a tag that exhibits a sequence for a desired intended purpose or application. One tag portion or tag domain is the “transposon end domain,” which tag portion or tag domain exhibits the transferred transposon end sequence. In some embodiments wherein the transferred strand also exhibits one or more other nucleotide sequences 5′-of the transferred transposon end sequence, the tag also has one or more other “tag domains” in said 5′-portion, each of which tag domains is provided for any desired purpose. For example, some embodiments comprise or consist of a transposon end composition that comprises or consists of: (i) a transferred strand that exhibits one or more sequences 5′-of the transferred transposon end sequence that comprises or consists of a tag domain selected from among one or more of a restriction site tag domain, a capture tag domain, a sequencing tag domain, an amplification tag domain, a detection tag domain, an address tag domain, and a transcription promoter domain; and (ii) a non-transferred strand that exhibits the non-transferred transposon end sequence. Certain embodiments of the method may use any one or more of said transposon end compositions.

In some embodiments, the discloses techniques are used to generate a nucleic acid library (e.g., a library 39) or a DNA fragment library, wherein the DNA fragment library comprises fragments of the target DNA having 5′ ends comprising sequences from transferred strands from transposon ends or transposon end compositions. In preferred embodiments, the sequences from the transferred strands comprise 5′ tag domains and in still more preferred embodiments, the DNA fragment library comprises fragments of target DNA comprising 3′ tags complementary to a transferred strand from a transposon end or transposon end composition. In some embodiments, the DNA fragment library comprises double-stranded fragments of the target DNA. The generated library can be used in sequencing reactions as provided herein.

The generated nucleic acid library may be sequenced according to any sequencing technique, such as those incorporating sequencing-by-synthesis methods described in U.S. Patent Publication Nos. 2007/0166705; 2006/0188901; 2006/0240439; 2006/0281109; 2005/0100900; U.S. Pat. No. 7,057,026; WO 05/065814; WO 06/064199; WO 07/010,251, the disclosures of which are incorporated herein by reference in their entireties. Alternatively, sequencing by ligation techniques may be used in the sequencing device. Such techniques use DNA ligase to incorporate oligonucleotides and identify the incorporation of such oligonucleotides and are described in U.S. Pat. Nos. 6,969,488; 6,172,218; and 6,306,597; the disclosures of which are incorporated herein by reference in their entireties. Some embodiments can utilize nanopore sequencing, whereby target nucleic acid strands, or nucleotides exonucleolytically removed from target nucleic acids, pass through a nanopore. As the target nucleic acids or nucleotides pass through the nanopore, each type of base can be identified by measuring fluctuations in the electrical conductance of the pore (U.S. Pat. No. 7,001,792; Soni & Meller, Clin. Chem. 53, 1996-2001 (2007); Healy, Nanomed. 2, 459-481 (2007); and Cockroft, et al. J. Am. Chem. Soc. 130, 818-820 (2008), the disclosures of which are incorporated herein by reference in their entireties). Yet other embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference in its entirety. Particular embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides, or with zeromode waveguides as described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference in their entireties. Other suitable alternative techniques include, for example, fluorescent in situ sequencing (FISSEQ), and Massively Parallel Signature Sequencing (MPSS). In particular embodiments, the sequencing may be performed via HiSeq, MiSeq, or HiScanSQ from Illumina (La Jolla, CA).

The transposome complexes 26 may be provided as pre-prepared compositions in which the active and inactive transposomes are already coupled to one another. In one embodiment, the transposome complexes 26 are provided as part a library preparation kit that may include additional elements, such appropriate primers for use in conjunction with the desired sequencing platform. The kit may include a transposome complex composition that includes only transposome complexes 26 that are all estimated to be within a particular size or weight tolerance. The sample preparation kit may also include one or more sample preparation enzymes, buffers, and/or reagents. The sample preparation kit may be provided as a prepackaged kit for preparing a library from a single sample or, in certain embodiments, may be provided as a multi-sample kit with a plurality of different available transposome complexes 26 of different sizes that can produce different library fragment lengths. The end user can select the desired length transposome complex 26 and proceed with the steps of library preparation using the selected size transposome complex 26. In another embodiment, the library preparation kit may permit the user to build or manufacture the transposome complex 26 according to the disclosed techniques from transposome monomers or dimers or from individual active and inactive transposomes.

The disclosed techniques may be used to prepare a nucleic acid library from a target nucleic acid (e.g., target nucleic acid 18). “Target nucleic acid” can be derived from any in vivo or in vitro source, including from one or multiple cells, tissues, organs, or organisms, whether living or dead, or from any biological or environmental source (e.g., water, air, soil). For example, in some embodiments, the target nucleic acid comprises or consists of eukaryotic and/or prokaryotic dsDNA that originates or that is derived from humans, animals, plants, fungi, (e.g., molds or yeasts), bacteria, viruses, viroids, mycoplasma, or other microorganisms. In some embodiments, the target nucleic acid comprises or consists of genomic DNA, subgenomic DNA, chromosomal DNA (e.g., from an isolated chromosome or a portion of a chromosome, e.g., from one or more genes or loci from a chromosome), mitochondrial DNA, chloroplast DNA, plasmid or other episomal-derived DNA (or recombinant DNA contained therein), or double-stranded cDNA made by reverse transcription of RNA using an RNA-dependent DNA polymerase or reverse transcriptase to generate first-strand cDNA and then extending a primer annealed to the first-strand cDNA to generate dsDNA. In some embodiments, the target nucleic acid comprises multiple dsDNA molecules in or prepared from nucleic acid molecules (e.g., multiple dsDNA molecules in or prepared from genomic DNA or cDNA prepared from RNA in or from a biological (e.g., cell, tissue, organ, organism) or environmental (e.g., water, air, soil, saliva, sputum, urine, feces) source. In some embodiments, the target nucleic acid is from an in vitro source. For example, in some embodiments, the target nucleic acid comprises or consists of dsDNA that is prepared in vitro from single-stranded DNA (ssDNA) or from single-stranded or double-stranded RNA (e.g., using methods that are well-known in the art, such as primer extension using a suitable DNA-dependent and/or RNA-dependent DNA polymerase (reverse transcriptase). In some embodiments, the target nucleic acid comprises or consists of dsDNA that is prepared from all or a portion of one or more double-stranded or single-stranded DNA or RNA molecules using any methods known in the art, including methods for: DNA or RNA amplification (e.g., PCR or reverse-transcriptase-PCR (RT-PCR), transcription-mediated amplification methods, with amplification of all or a portion of one or more nucleic acid molecules); molecular cloning of all or a portion of one or more nucleic acid molecules in a plasmid, fosmid, BAC or other vector that subsequently is replicated in a suitable host cell; or capture of one or more nucleic acid molecules by hybridization, such as by hybridization to DNA probes on an array or microarray.

In alternative embodiments, the transposome complex may include multiple transposomes that are coupled or linked to a solid surface or substrate, thereby forming a surface-linked transposome complex (also referred to herein as SLTC). For example, the substrate may be a magnetic bead, and thus multiple transposomes coupled to the magnetic bead may be referred to as bead-linked transposome complexes (BLTs). However, it should be understood that, while certain embodiments are discussed in the context of beads, other substrates are also contemplated, including planar surfaces, patterned surfaces, shaped surfaces, etc. Further, in certain embodiments, the disclosed linked transposome complexes may be used in solution-based reactions. The surface-linked transposome complexes may include transposomes as generally discussed herein.

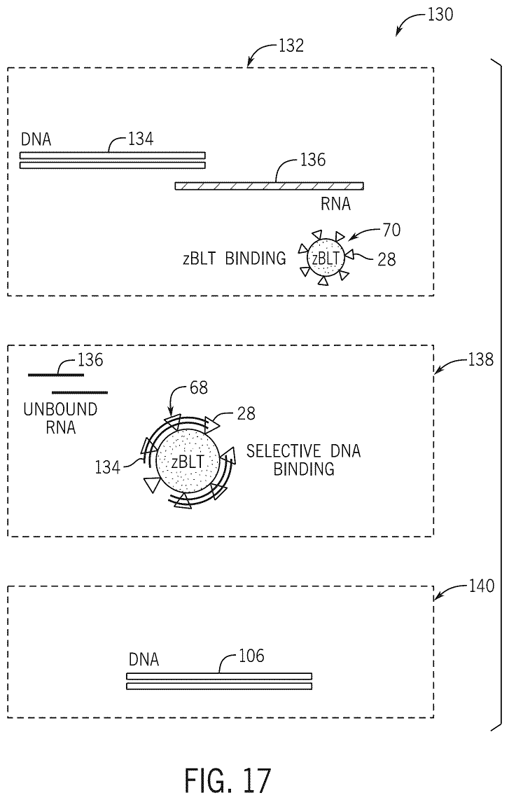

The transposomes of the surface-linked transposome complex may include one or more inactive transposomes, one or more active transposomes, or a combination of both inactive transposomes and active transposomes. For example, each of the transposomes of the surface-linked transposome complex may be inactive transposomes. As another non-limiting example, each of the transposome of the surface-linked transposome complex may be active transposomes. As discussed herein, the active transposomes are catalytically active (e.g., the transposomes are not inactivated due to chemical modifications or heat), and thus are capable of inserting into a target nucleic acid. An “inactive” transposome, or a “zombie transposome”, refers to a catalytically inactive transposome that is unable to join (e.g., insert via ligation) into a target nucleic acid, but the inactive transposome nonetheless binds to the target nucleic acid. That is, an inactive transposome or zombie transposome is capable of binding a target DNA but unable to catalyze the joining of the target DNA and the transposon-bound sequences. At least in some instances, the inactive transposome may be inactive due to inactivation of the transposase of the transposome, such as by modifying an amino acid sequence of the transposase. It is presently recognized that a surface-linked transposome complex that includes inactive transposomes may enable tuning of the insert size distribution of the sequencing library produced using the surface-linked transposome complex. Further, in embodiments where the surface-linked transposome complex includes both inactive transposomes and active transposomes, the inactive transposomes may modulate the position of tagmentation events. Furthermore, the surface-linked transposome complexes that include inactive transposomes may facilitate the separation and/or purification of nucleic acids, as described in further detail with respect to FIGS. 17-19.

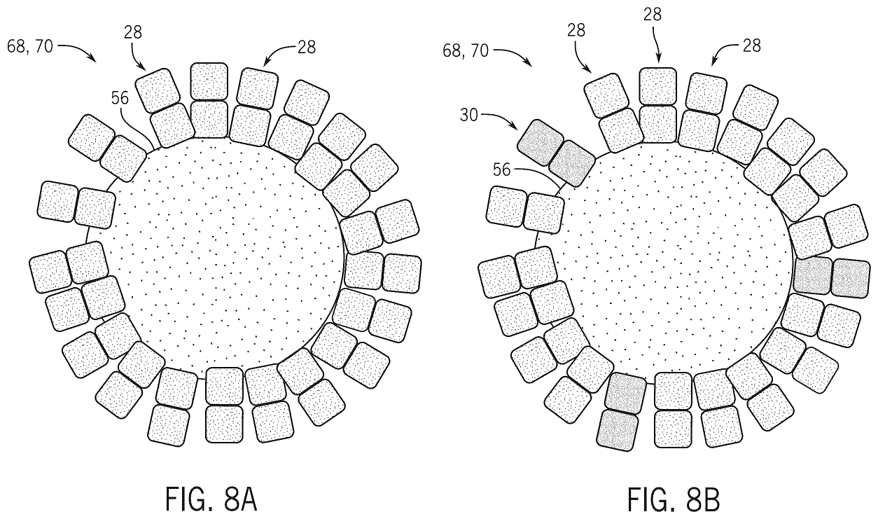

To illustrate a first example of a surface-linked transposome complex, FIG. 8A shows a schematic diagram of a surface-linked transposome complex 68 where each transposome is an inactive transposome 36 that is coupled to (e.g., directly or indirectly coupled to) a substrate 56 (e.g., a magnetic bead). Accordingly, the surface-linked transposome complex 68 of FIG. 8A may be referred to as an inactive bead-linked transposome complex (zBLT) 70a or an inactive surface-linked transposome complex (zSLTC). That is, a zBLT or zSLTC refers to a transposome complex that includes one or more catalytically inactive transposomes. In this illustrated embodiment of the zBLT 70a of FIG. 8A, all of the transposomes are inactive transposomes 28. However, as discussed below with respect to FIG. 8B, a first portion of the transposomes may be inactive transposomes 28 while a second portion may include action transposomes 30.

Referring to FIG. 8A, each inactive transposome 28 of the SLTC 68 may be coupled to a linker (not shown) such that each inactive transposome 28 may be individually linked to the surface of the magnetic bead (e.g., substrate 56). In general, the linker between the magnetic bead and the inactive transposome 28 and/or active transposome 30, as discussed in FIG. 8B, may be a binding due to a functional groups on the surface of the beads, such as a carboxyl group, a hydroxyl group, or a carbonyl group, and the binding may be facilitated based on a concentration of salts present in solution. In some embodiments, the inactive transposomes 28 may be coupled to one another (e.g. via an adaptor). In some embodiments, a portion of the inactive transposomes 28 may include a linker while a remaining portion of the inactive transposomes 28 may not include the linker. For example, the substrate 56 may be directly coupled to a first portion of inactive transposomes 28 and indirectly coupled to a second portion of inactive transposomes 28. That is, the first portion of the inactive transposomes 28 may be directly coupled to the substrate 56 via a linker of each of the inactive transposomes 28 of the first portion. The second portion (e.g., different from the first portion) of inactive transposomes 28 may be directly coupled to the first portion of inactive transposomes 28 via an adaptor. As such, the second portion of inactive transposomes 28 are indirectly coupled to the substrate 56 via a linker of the first portion and the inactive transposomes 28. It should be noted that, while the illustrated embodiment of the substrate 56 in FIG. 8A shows a magnetic bead (e.g., a bead), other suitable solid surfaces may be used. For example, the substrate 56 may include an array of wells and a transposomes (e.g., inactive transposomes 28, active transposomes 30, or both) may be coupled to a surface of a well. Further, it should be noted that one or more of the inactive transposomes 28 of the SLTC 68 or zBLT may differ structurally from adjacent or neighboring transposomes due to a different end sequence and/or linking sequence that couples the inactive transposomes 28 together, i.e., to neighboring transposomes. That is, the SLTC 68 or zBLT 70a may include the inactive transposome 32, 34, 36, or other types of inactive transposomes.

In some embodiments, a mixture of both active transposomes 30 and inactive transposomes 28 may be coupled to the substrate 56. To illustrate a second example of a SLTC 68, FIG. 8B shows a schematic diagram of a zBLT 70b (e.g., the SLTC 68) that includes both inactive transposomes 28 and active transposomes 30 that are coupled to a magnetic bead (e.g., a bead-linked transposome complex). Accordingly, the SLTC 68 of FIG. 8B may be referred to as a zBLT 70b that is active. As used herein, an “active” SLTC, BLT, zBLT, or zSLTC refers to a SLTC, BLT, zBLT, or zSLTC that includes active transposomes 30. As used herein, a SLTC 68 may generally refer to a surface-linked transposome complex that is either active or inactive. Further, a zBLT refers to a bead-linked transposome complex where at least one transposome is an inactive transposome 28.

In a generally similar manner as described above with respect to the SLTC 68 or zBLT 70a of FIG. 8A, each of the active transposomes 30 and/or inactive transposomes 28 of the zBLT 70b may have its own linker (not shown) such that each of the active transposomes 30 and/or inactive transposomes 28 may be individually linked to the surface of the bead. In some embodiments, the active transposomes 30 and/or inactive transposomes 28 may be coupled to one another (e.g. via an adaptor). In some embodiments, a portion of the transposomes (e.g., a portion of the active transposomes, the inactive transposomes, or both) may include a linker while a remaining portion of the transposomes may not include the link. Furthermore, although the illustrated embodiment includes the inactive transposome28, it should be noted that one or more of the inactive transposomes 28 of the zBLT 70b may differ structurally from adjacent or neighboring transposomes due to a different end sequence and/or linking sequence that couples the inactive transposomes 28 together, i.e., to neighboring transposomes. That is, the zBLT 70b may include the inactive transposome 32, 34, 36, or other types of inactive transposomes. For example, the surface-linked transposome complex 68 may include an initiator transposome 32 that forms a seed from which the transposome complex 26 is grown and various linker transposomes 34, 36 that may include sequences that are complementary to one another and/or the initiator transposome 32.

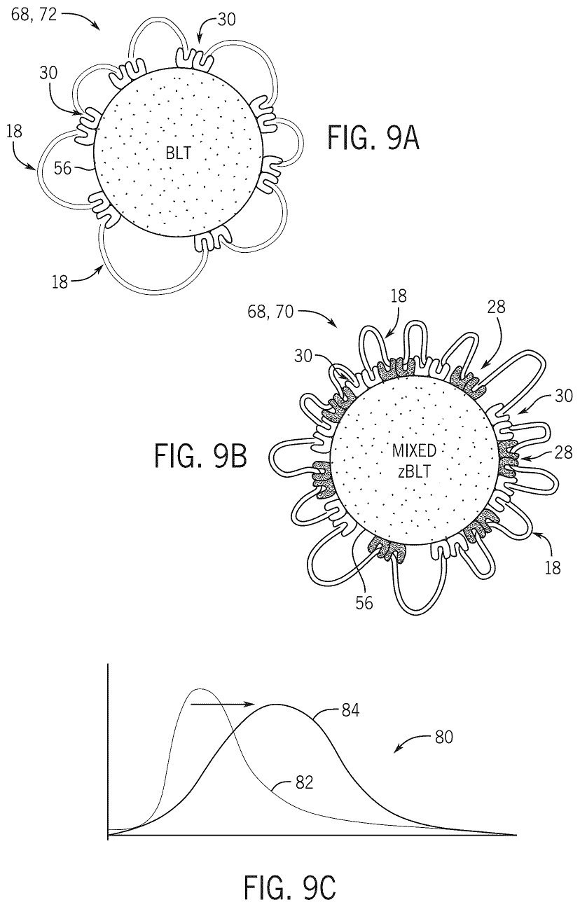

It is presently recognized that the fragment or insert size produced by SLTCs 68 including both active transposomes 30 and inactive transposomes 28 (e.g., the zBLT 70b shown in FIG. 8B) may be tuned based on the relative number of inactive transposomes to active transposomes. To illustrate this, FIGS. 9A-9C (i.e., FIG. 9A, FIG. 9B, and FIG. 9C) shows schematic diagrams of two examples of surface-linked transposomes including active transposomes 30.

More specifically, FIG. 9A shows a schematic diagram of a first surface-linked transposome complex 68 (e.g., a BLT 72) including active transposomes 30 bound to a target nucleic acid 18. That is, all of the transposomes of the BLT 72 shown in FIG. 9A are active transposomes. As shown in the illustrated embodiment, the active transposomes 30 are distributed radially about the substrate 56. In some embodiments, each of the active transposomes 30 may be distributed evenly about the substrate 56 (e.g., a magnetic bead) such that the active transposomes 30 are substantially equidistant from a neighboring active transposome 30. In other contemplated arrangements, each of the active transposomes 30 can be distributed on the substrate 56 in a patterned arrangement that is not necessarily equidistant. In the illustrated embodiment, the substrate 56 does not include any surface-linked inactive transposomes 28. Thus, the fragment length of the bound nucleic acid 18 is a function of the position and spacing of the active transposomes 30 on the substrate 56. This spacing and/or positioning can be based on printing, patterning or other techniques for activating the substrate 56.

FIG. 9B shows a schematic diagram of a second surface-linked transposome complex 68 (e.g., a zBLT 70b) including both active transposomes 30 and inactive transposomes 28 bound to a target nucleic acid 18. As shown in the illustrated embodiment, the active transposomes 30 are distributed radially about the substrate 56 (e.g., a magnetic bead). In some embodiments, each of the active transposomes 30 may be distributed evenly about the substrate 56 (e.g., a magnetic bead) such that the active transposomes 30 are substantially equidistant from a neighboring active transposome 30. Further, as shown in the illustrated embodiment, the number of inactive transposomes 28 between two neighboring active transposomes 30 is substantially the same for each pair of active transposomes 30. Providing a uniform distribution of active transposomes 30 and inactive transposomes 28 may facilitate generating more uniform fragments from the target nucleic acid 18.

FIG. 9C shows a graph 80 illustrating the size distribution of fragments of the target nucleic acid 18 produced from the BLT 72 and the zBLT 70b. The curve 82 corresponds to the size distribution of fragments produced using the BLT 72. The curve 84 corresponds to the size distribution of fragments produced using the zBLT 70b. As shown in the graph 80, the size distribution of fragments (e.g., fragments 22 as described with respect to FIG. 1) produced from the target nucleic acid 18 using the zBLT 70b are relatively longer as compared to the size distribution of fragments produced from the target nucleic acid 18 using BLT 72.

At least in some instances, it may be advantageous to control the size of fragments (e.g., fragments 22 as described with respect to FIG. 1) produced from a target nucleic acid 18. It is presently recognized that the size distribution may be modified (e.g., the average size of fragments may be increased or decreased) by tuning the number of active transposomes 30 and inactive transposomes 28 and/or the ratio of the number of active transposomes 30 and inactive transposomes 28 that are bound or linked to the substrate 56 of the SLTC 68 (e.g., zBLT 70b of FIG. 8B). For example, a lower transposome loading of active transposomes 30 may produce relatively larger fragments and may further reduce the amount of sample input that would be processed by the SLTC 68 as compared to a SLTC 68 having a relatively higher transposome loading of active transposomes 30. The inactive transposomes 28 may sterically block a portion of nucleic acid 18 (e.g., a dsDNA sample) from tagmentation, and thus increase the resulting fragment size. Accordingly, tuning the relative proportion or the number of active transposomes 30 and/or inactive transposomes 28 of an SLTC 68 consisting of active transposomes 30 and inactive transposomes 28 may vary the fragment size. Further, the inactive transposomes 28 may increase the DNA binding capacity (e.g., the number of locations where a target nucleic acid 18 is bound to a SLTC 68) of each SLTC 68. Accordingly, tuning the number of inactive transposomes 28 may provide an additional mechanism to prevent nucleic acid (e.g., DNA or RNA) dissociation from the SLTC 68 due to hydrodynamic forces that occur during library preparation. It should be noted that controlling the DNA binding capacity may be advantageous for producing relatively large fragments using the SLTC 68, which may otherwise have few attachment points to magnetic beads 56 if it were only mediated by active transposomes.

In an embodiment, the active transposomes 30 make up less than half of the total transposomes (e.g., the active transposomes 30 and inactive transposomes 28) of the mixture distributed on a particular surface. In an embodiment, the active transposomes 30 are less than 20%, less than 15%, less than 10%, or less than 5% of the mixture. In an embodiment, the active transposomes 30 represent between 0.1%-20%, between 1%-5%, between 5%-10%, between 10%-15%, between 15%-20%, between 20%-30%, or between 30%-50% of the mixture.

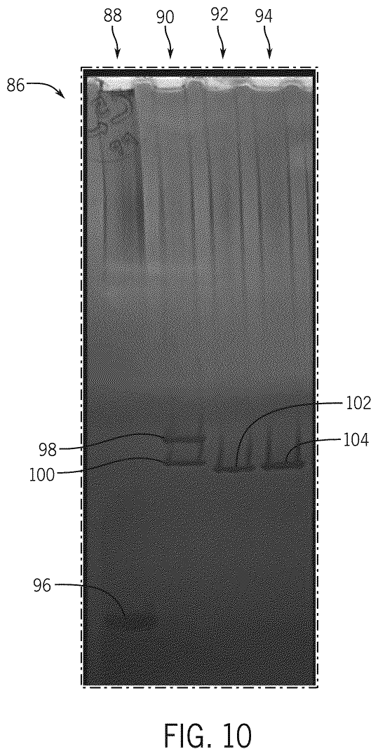

FIG. 10 shows gel electrophoresis results illustrating transposome formation. More specifically, FIG. 10 shows an image 86 of blue native protein gel results that illustrates whether dimeric transposome formed successfully using the altered transferred strand oligonucleotide. The gel results show four lanes 88, 90, 92, and 94. Lane 88 shows results 96 (i.e., a band) of a BSA marker. Lane 90 shows results 98, 100 (i.e., bands) of a transposome mixture consisted of two transposomes with different transposon DNA lengths. Lane 92 shows results 102 (i.e., a band) of bead-linked transposomes with inactive transposomes 28 built with 3′ phosphorylated transferred strand 102. Lane 94 shows results 104 (i.e., a band) of a transposome built with transferred strand of the same size as in lane 92 lacking 3′ phosphate block. Comparison of control transposomes, including a mixed formulation of active transposomes (a mixture of two active forms of transposome with different transposon DNA lengths) and a transposome with a short transposon (e.g., 19 bp) shows a dimeric transposome complex of the expected size, indicating that 3′phosphate modification did not interfere with formation of the dimeric transposome complex.

FIG. 11 shows a first graph 106 illustrating tagmentation activity of a BLT 72 (e.g., ‘Active BLT’) and tagmentation activity of a zBLT. More specifically, FIG. 11 shows fluorescence resonance energy transfer (FRET) tagmentation activity assays illustrating undetectable tagmentation activity with bead-linked transposomes including inactive transposomes 28 (right) as compared to bead-linked transposomes including active transposomes 30 (left).

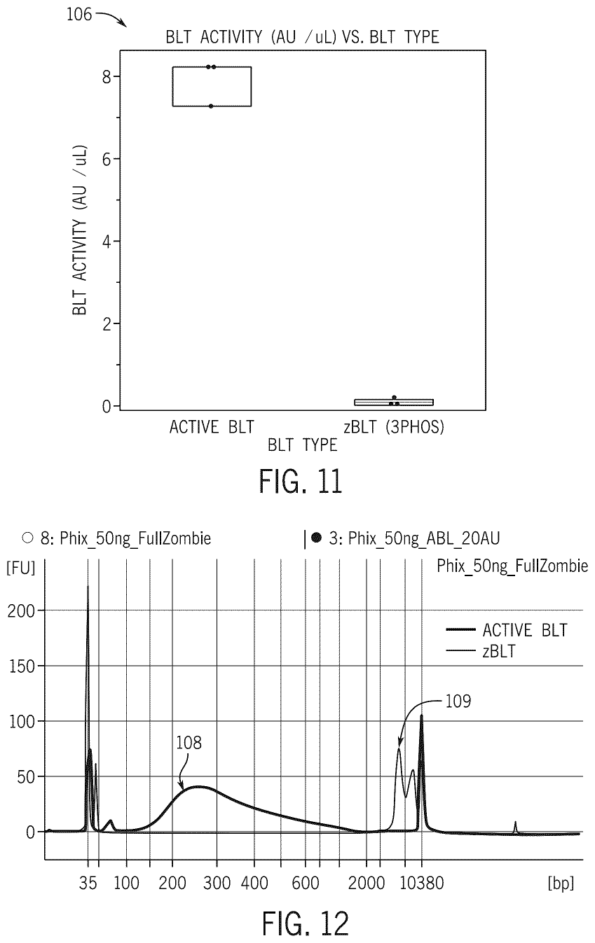

FIG. 12 shows a second graph illustrating a curve 108 corresponding to tagmentation treatment of a BLT 72 (e.g., Active BLT) and curve 109 corresponding to tagmentation treatment of a BLT 70 containing inactive transposomes (e.g., the zBLT 70a of FIG. 8A). More specifically, FIG. 12 shows bioanalyzer traces (e.g., curves 108 and 109) of PhiX DNA subjected to tagmentation conditions with BLT 72 including active transposomes 30 (e.g., curve 108) or zBLT 70a including inactive transposomes (e.g., curve 109) High molecular weight fragments in zBLT trace are consistent with unfragmented PhiX (˜5 KB).

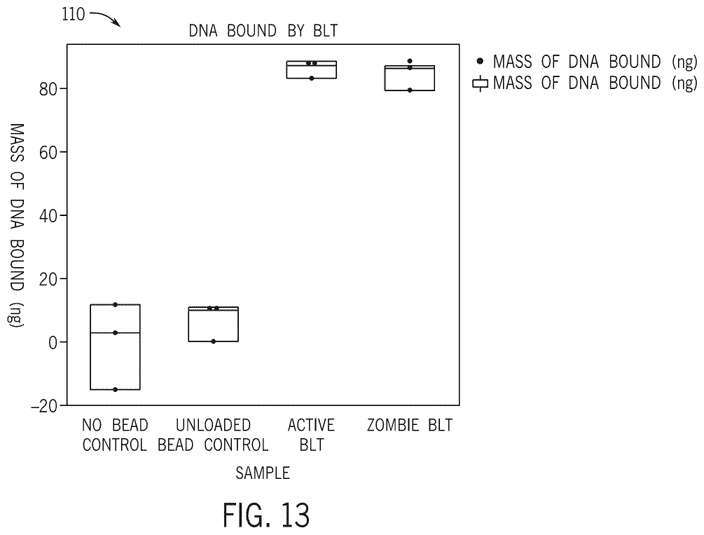

FIG. 13 shows a graph 110 illustrating an amount of nucleic acid bound to BLTs 70a and 72. More specifically, the graph 110 illustrates a measured amount of DNA obtained using a control without a BLT 72 (e.g., ‘No bead control’), a control bead (e.g., streptavidin bead) that does not include attached transposomes and/or is not loaded with DNA (e.g., ‘Unloaded bead control’), a BLT 72 (e.g., ‘Active BLT’, such as the BLT 72 described with respect to FIG. 9A), and a zBLT 70a (e.g., ‘Zombie BLT). To test whether the zBLT 70a had DNA binding activity, a DNA binding assay was performed. The control bead, the BLT 72 (i.e., having bound DNA), and the zBLT 70a were each incubated with lambda genomic DNA under standard tagmentation conditions. After tagmentation, the concentration of DNA remaining in the supernatant was measured via a fluorescent DNA binding dye (e.g., via a Qubit Fluorometer). The measurement was used to calculate the amount of DNA bound by the BLT 72 or zBLT 70a. The results in graph 110 show that the zBLT 70a bound similar amounts of DNA as the BLT 72. The control beads showed little non-specific binding, indicating that the inactive transposome 28 was responsible for this DNA binding activity.

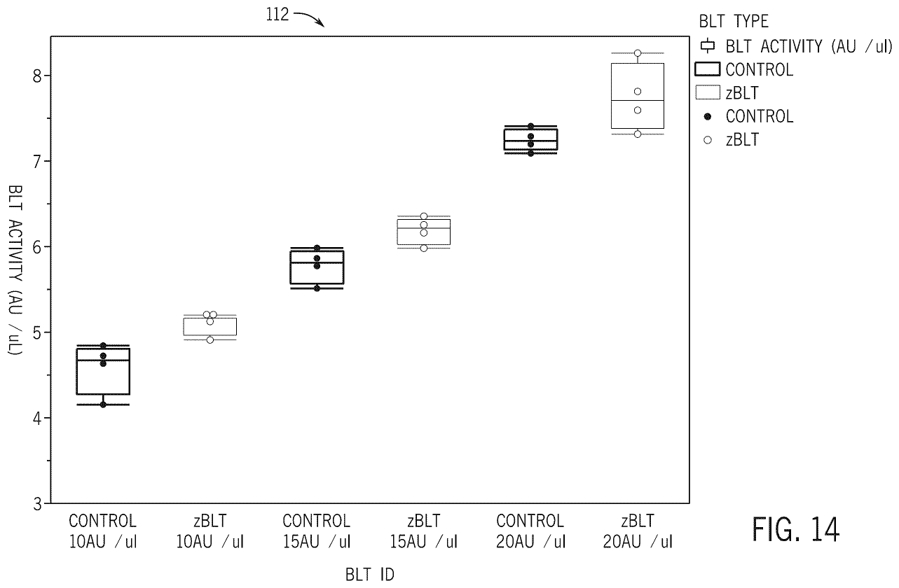

FIG. 14 shows a graph 112 illustrating tagmentation activity of a BLT 72 and tagmentation activity of a zBLT 70b for different mixtures or ratios of active transposomes 30 and inactive transposomes 28. Using inactive transposomes, zBLTs 70b or SLTCs with mixtures of active transposomes 30 and inactive transposomes 28 may be built with a stepwise procedure for binding transposome to beads. For example, BLTs or SLTCs may be generated by providing active transposomes to the substrate 56, optionally washing or removing excess non-bound active transposomes, and subsequently adding inactive transposomes. At least in some instances, the inactive transposomes 28 and active transposomes 30 may be added to the bead as described with respect to FIG. 5.

In the example corresponding to FIG. 14, the active transposomes 30 were bound to beads (e.g., substrate 56 or magnetic bead) at the desired activity density (e.g., amounts of active transposomes 30 and inactive transposomes 28 that provide a DNA binding capacity). After removal of excess non-bead bound active transposomes 30 from the supernatant, the desired concentration of inactive transposomes 28 were added. The inactive transposome 28 bind to the substrate 56 to produce BLTs 72 including both active transposomes 30 and inactive transposomes 28. It should be noted that the addition of active transposomes 30 and, subsequently, inactive transposomes 28 during the build processes may facilitate the non-competitive binding by the active transposomes 30 to the beads noncompetitively, which may provide a desired activity by the BLT 72. Table 1 shows examples of zBLTs generated in accordance with the disclosure techniques. In addition to the zBLTs 70b in Table 1, pure active BLTs (e.g., active SLTCs) were also prepared at 10, 15, 20, and 40 AU/μl as controls. FRET testing of select BLTs revealed that sequential transposome binding achieves equivalent activity of zBLTs compared with the pure active-only equivalent. FRET activity results of zBLTs and the pure active-only equivalent BLTs.

TABLE 1
Bead-linked transposomes generating
using stepwise transposome binding.

| | | Activity | Amount |
|---|---|---|---|
| | | of active | of inactive |
| | | transposome | transposome |
| | | (AU/μl)11 | (fmol/μl) 12 |
| BLT ID | BLT Description | (1st binding event) | (2nd binding event) |
| 10 AU/μl | 10 AU/μl active | 10 | 830 |
| zBLT | transposome | | |
| | followed by | | |
| | 4x volume | | |
| | inactive | | |
| | transposome | | |
| 15 AU/μl | 15 AU/μl active | 15 | 726 |
| zBLT | transposome | | |
| | followed by | | |
| | 2.3x volume | | |
| | inactive | | |
| | transposome | | |
| 20 AU/μl | 20 AU/μl active | 20 | 623 |
| zBLT | transposome | | |
| | followed by | | |
| | 1.5x volume | | |
| | inactive | | |
| | transposome | | |
| 40 AU/μl | 40 AU/μl ABL | 40 | 456 |
| zABL | transposome | | |
| | followed by | | |
| | 0.5x volume | | |
| | inactive | | |
| | transposome | | |

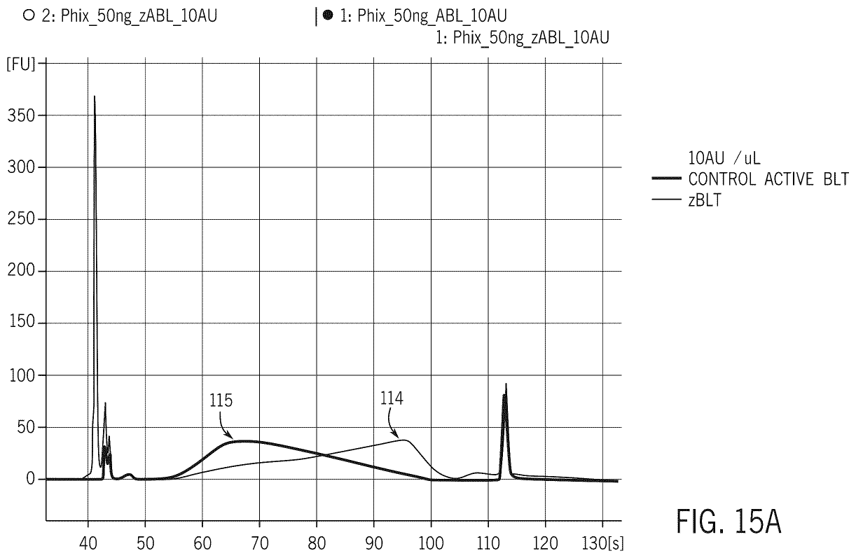

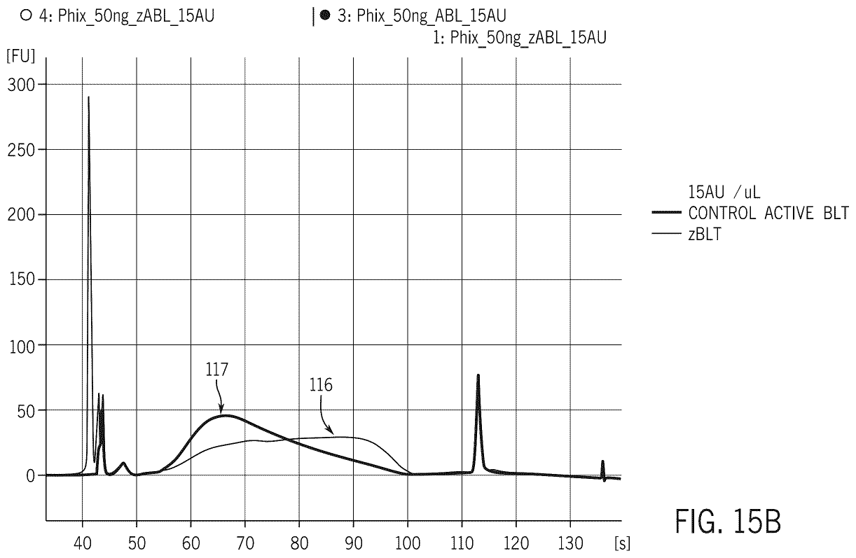

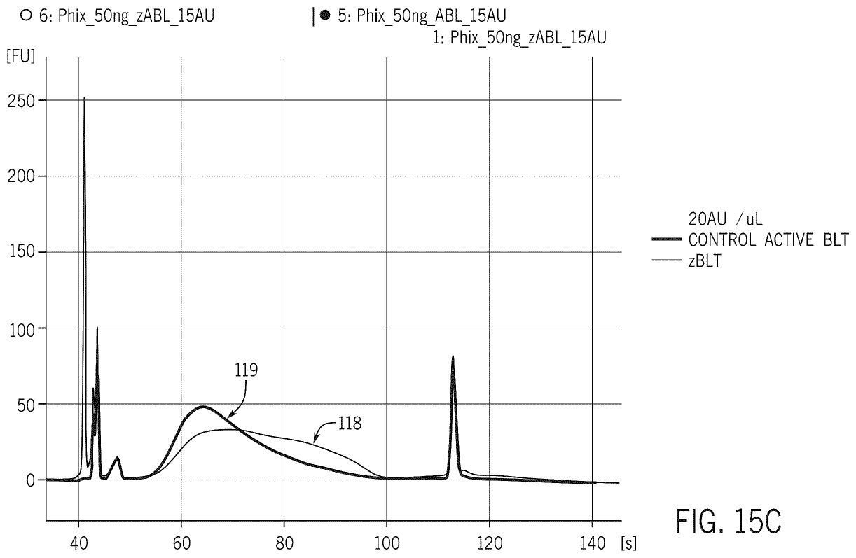

FIGS. 15A-15C (i.e., FIGS. 15A, 15B, and 15C) illustrate the fragment size distribution for zBLTs 70b compared to pure active BLT 72 controls (e.g., BLTs or SLTCs that do not include inactive transposomes 28). Electrophoretic analysis of tagmentation products via Bioanalyzer show that the addition of inactive transposomes led to shifts in the fragment size distribution towards larger fragment sizes compared to the control BLTs 72. For example, FIG. 15A shows a first graph illustrating a curve 114 corresponding to tagmentation activity of a zBLT with a 10 AU/μL inactive transposomes 28 and a curve 115 corresponding to tagmentation activity of the pure active BLT. Further, FIG. 15B shows a second graph illustrating a curve 116 corresponding to tagmentation activity of a bead-linked transposome complex with 15 AU/μL of inactive transposomes 28 and a curve 117 corresponding to tagmentation activity of the pure active BLT. FIG. 15C shows a third graph illustrating a curve 118 corresponding to tagmentation activity of a BLT with 20 AU/μL of inactive transposomes 28 and a curve 115 corresponding to tagmentation activity of the pure active BLT. In general, the graphs of FIGS. 15A, 15B, and 15C illustrate that addition of inactive transposomes 28 to BLTs 72 with active transposome 30 leads to shifts in observed fragment size. Samples tagmented by BLTs 72 (e.g., corresponding to curves 115, 117, and 119) compared with samples tagmented by zBLTs 70b (e.g., corresponding to curves 114, 116, and 118) show a shift towards fragments with larger sizes for the BLTs 72 including inactive transposomes 28. Accordingly, FIGS. 15A-15C show techniques for shifting fragment or insert size via addition of inactive transposomes to a SLTC 68 having active transposomes 30.

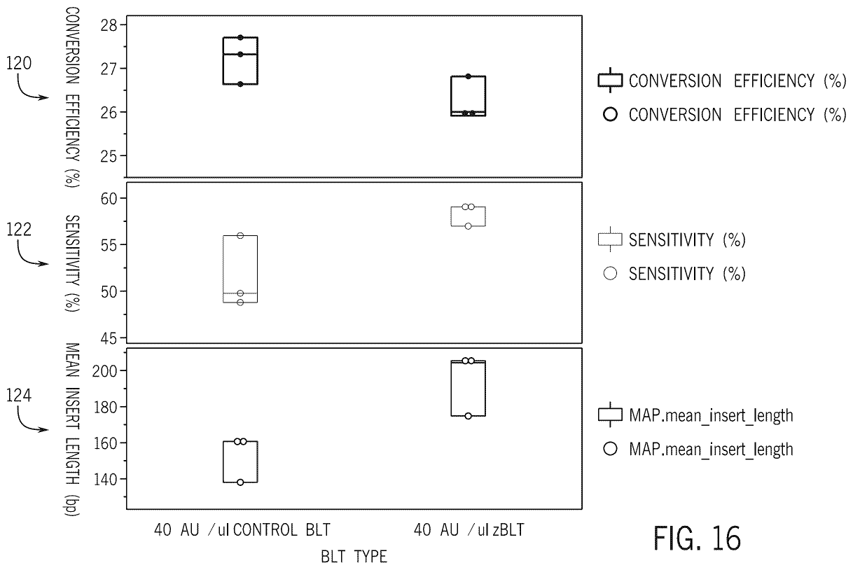

To characterize the performance of mixed zBLTs 70 (e.g., the zBLTs 70b as described with respect to FIG. 8B) in a sequencing assay, the 40 AU/μL pure active BLT (e.g., represented in Table 1) and 40 AU/μL zBLTs were used to prepare libraries from a 10 ng sample of 1% NA12877 in NA12878 background for enrichment and sequencing. The pure active BLTs and the zBLTs provided similar library conversion efficiency and somatic variant calling sensitivity, as shown in FIG. 16. These metrics were maintained while seeing a shift in insert size of approximately 50 bp. FIG. 16 shows a first graph 120 illustrating conversion efficiency, a second graph 122 illustrating sensitivity, and a third graph 124 mean insertion length for a BLT 72 (e.g., an active BLT) and a zBLT 70b. More specifically, the graphs show performance evaluation of zBLT 70 compared to a control BLT 72 in sequencing assay using a 10 ng input of NA12877 in NA12878 background.

As described herein, an SLTC 68 may be used to separate a mixture of different types of nucleic acids. For example, a zSLTC or zBLT 70a that does not include catalytically active transposomes (e.g., active transposomes 30), such as the zBLT 70a described with respect to FIG. 8A, may bind to nucleic acids, while not tagmenting the nucleic acids. To illustrate this, FIG. 17 shows a flow diagram of a method 130 for separating a mixture of different types of nucleic acids (e.g. DNA and RNA) using a zBLT 70a. At block 132, a zBLT 70a is provided to a mixture of nucleic acids (e.g., a first nucleic acid 134 (e.g., DNA) and a second nucleic acid 136 (e.g., RNA)). In the illustrated embodiment, the zBLT 70a includes inactive transposomes 32. As discussed herein, inactive transposomes 28 may be capable of binding a nucleic acid however the inactive transposomes are catalytically inactive. That is, the inactive transposomes 28 do not tagment and cleave nucleic acids. In the example method 130, the zBLT 70 may selectively bind the first nucleic acid 134, but not the second nucleic acid 136. Accordingly, at block 138, the zBLT 70a may bind to the first nucleic acid 134 via the adaptors while the second nucleic acid 136 remains unbound (e.g., in solution). At block 140, the second nucleic acid 136 may be removed from a solution containing the zBLT 70a bound to the first nucleic acid 134 via washing and elution step(s). In this way, the zBLT 70a may facilitate the separation of different types of nucleic acids present within a solution. At least in some instance, utilizing the zBLT 70a to separate the first nucleic acid from the second nucleic acid may improve the retention of the second nucleic acid that may be lost or otherwise damaged during certain separation techniques. Furthermore, it is presently recognized that separation using zBLTs 70a may be relatively easier than techniques using reagents used in the extraction of DNA, RNA, or proteins, such as trizol or chloroform.

Although the discussion of FIG. 17 above generally describes separating DNA from RNA, it should be noted that an SLTC 68, such as a zBLT 70a may be used to separate other components. For example, the zBLT 70a may be used to separate double-stranded DNA from single-stranded RNA in a generally similar manner as described above with respect to FIG. 17. Further, the zBLT 70 may be used to separate double-stranded DNA from single-stranded DN. Furthermore, the zBLT 70a may be used to separate or extract double-stranded DNA from a mixture of other types of nucleic acids, proteins, and organic matter (lipids, carbohydrates, etc.).

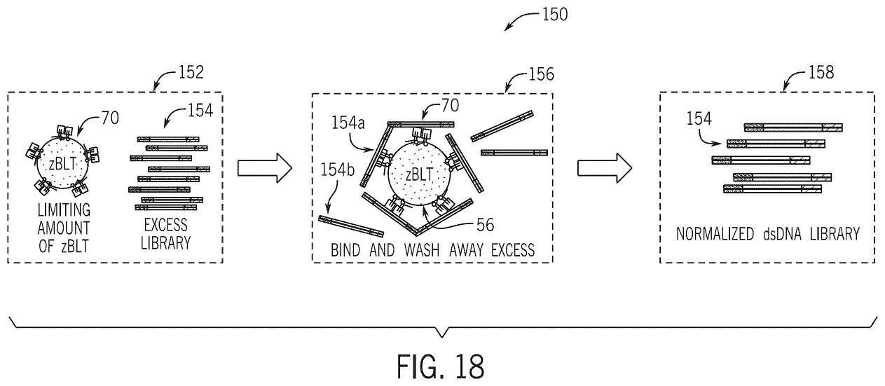

In some embodiments, the SLTC 68 facilitate normalizing amounts of nucleic acids across different samples. For example, a zSLTC or zBLT 70a that does not include catalytically active transposomes may bind to an amount of nucleic acids based on the nucleic acid binding capacity of the zBLT 70a imparted by the amount of inactive transposomes 28 bound to the substrate 56 of the zBLT 70a. To illustrate this, FIG. 18 shows a flow diagram of a method 150 for normalizing amounts of nucleic acids across different samples. At block 152, a zBLT 70a is provided to a solution containing nucleic acids 154. In general, the nucleic acids 154 may include nucleic acids or nucleic acid fragments. It should be noted that, tuning the ratio of the zBLTs 70a or zSLTC to the nucleic acid 154 may facilitate separating a uniform amount of nucleic acid or nucleic acids fragments across multiple samples. For example, the ratio of the SLTC 68 or zBLT 70a to the nucleic acids 154 where the nucleic acids 154 are in excess may facilitate producing a uniform amount of nucleic acid fragments. When the zBLTs 70 are of a same type or all have a predictable binding capacity for DNA, based on having a generally same or similar distribution of inactive transposomes 28 distributed on the surface relative to one another, then the amount of DNA predicted to bind per bead can be used to normalize DNA between samples.

At block 156, the zBLT 70a bind at least a portion of the nucleic acids or nucleic acid fragments to form a nucleic acid-surface linked transposomes complex. In the illustrated embodiment, the zBLT 70a is bound to a first portion 154a of nucleic acids 154 while a second portion 154b of the nucleic acids 154 remain in the solution unbound. As such, the first portion 154a may be separated from the second portion 154b via the magnetic bead (e.g., substrate 56) of the zBLT 70a. The second portion 154b of nucleic acid 154 that is not coupled to the substrate 56 of the zBLT 70a may be washed away. After removing the unbound nucleic acid, at block 158, the first portion 154a may be retrieved. Accordingly, the method 150 may be repeated for multiple samples of nucleic acids 154 to produce a normalized amount of nucleic acid for each sample. That is, the method 150 may be applied to a first sample having a first amount of a first nucleic acid and second sample having a second amount of a second nucleic acid. For each sample, the amount of nucleic acid may be in excess to the surface bound transposomes complex to capture, using the beads, a uniform amount of nucleic acid for each of the samples. Accordingly, after applying the techniques described in the method 150, a first portion of the first amount of nucleic acid and a second portion of the second amount of nucleic acid may be retrieved. By tuning the amount of zBLT 70a to the amount of nucleic acid, the first portion and the second portion may be substantially equal (e.g., within 1%, 5%, or 10% of one another as estimated by DNA concentration). Additionally or alternatively, the method 150 may be used to standardize the concentration of a sample to provide a desired or optimal (e.g., based on a limit of detection of a device) loading concentration for sequencing. For example, the normalization may be used to standardize the concentration of a pooled sample.

It should be noted that the method 150 may be used to normalize DNAfragments. Moreover, normalization with the zBLT 70a may improve the speed of normalization as compared to certain conventional techniques (e.g., manual normalization). For example, the normalization with zBLTs 70a may enable certain steps of manual normalization to be omitted (e.g., quantifying individual samples, running size analysis of individual samples, or normalizing samples that have different volumes). In this way, the disclosed techniques may improve the speed of normalization of nucleic acid fragments.

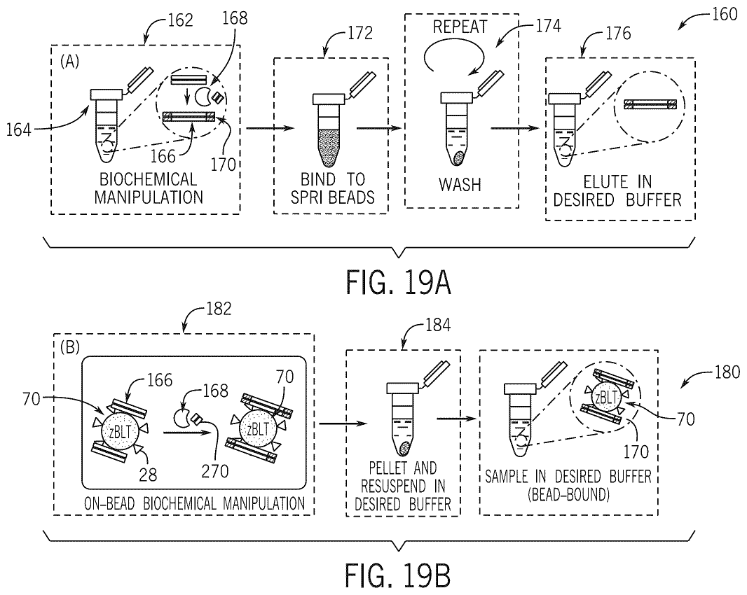

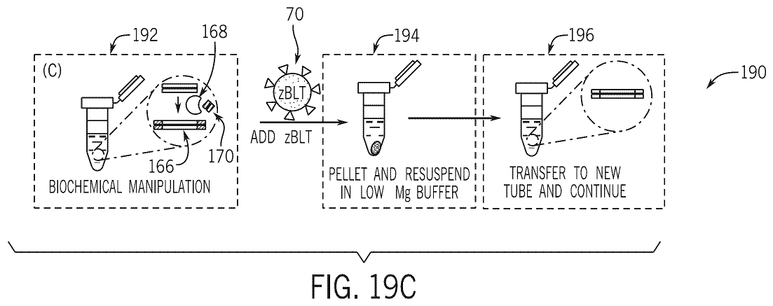

In some embodiments, the SLTC 68 may facilitate buffer exchange. For example, the zBLT 70a may capture a nucleic acids in a first solution and the capture nucleic acids may be transferred to and suspended in a second solution. To illustrate this, FIGS. 19A-19C (e.g., FIG. 19A, FIG. 19B, and FIG. 19C generally illustrate techniques for buffer exchange.

FIG. 19A shows a flow diagram of a method 160 for performing buffer exchange using a magnetic bead. At block 162, a vessel 164 (e.g., a centrifuge tube) including a nucleic acid sample 166 is provided. It is presently recognized that certain biochemical manipulation techniques (e.g., reactions involve an enzyme 168 that may add or remove an adaptor 170 to the nucleic acid sample 166) may react more efficiently under certain buffer conditions. Accordingly, it may be advantageous to transfer the nucleic acid sample 166 to a solution having a different buffer. At block 172, magnetic beads (e.g., solid phase reversible immobilization (SPRI beads)) are added to the solution in the vessel 164. The magnetic beads may be paramagnetic particles having carboxyl groups that reversibly bind the nucleic acid sample in the vessel 164. The magnetic beads may rely on charged interactions and a crowding agent to drive binding of nucleic acids non-specifically to a magnetic bead. For example, a salt-PEG solution or 80% ethanol solution may be used to suspend the SPRI beads to maintain interaction between the nucleic acids and beads during purification. After the magnetic beads bind the nucleic acid, multiple wash cycles may be implemented, at block 174. After a sufficient number of washing cycles (e.g., the step at block 174) are performed, the nucleic acids may be eluted in a desired buffer, a block 176.

It is presently recognized that it may be more efficient, and less time-consuming, to utilize the zBLT 70a having inactive transposomes 28 to facilitate the buffer exchange instead of the magnetic beads described with respect to the method 160. For example, utilizing the zBLTs 70a may reduce the number of wash cycles. To generally illustrate this, FIG. 19B shows a flow diagram of a method 180 for performing buffer exchange using a zBLT 70a during a biochemical manipulation of a nucleic acid sample 166.

At block 182, a zBLT 70a bound to a nucleic acid sample 166 may be provided with an enzyme 168. In the illustrated embodiment, the enzyme 168 is capable of adding an adaptor 170 (e.g., an end sequence) to the nucleic acid sample 166. It is presently recognized that the zBLT 70a may bind the nucleic acid sample 166 such that the enzyme 168 may be capable of performing the biochemical manipulation (e.g., adding the adaptor 170) while the nucleic acid sample 166 is bound to the zBLT 70a. Accordingly, this may reduce the amount of time by reducing the number of steps for performing buffer exchange. At least in some instances, the zBLT 70a may preferentially bind certain nucleic acids over other nucleic acids. For example, the zBLT 70a may have a relatively higher affinity for binding DNA as compared to RNA. At least in some instances, a salt may facilitate the transposome-nucleic acid complex formation. For example, the salt may include a divalent cation, such as Mg²⁺. At block 184, the zBLT 70a including the nucleic acid sample 166 may be centrifuged or subject to a magnetic field (e.g., via magnetic separation) to form a pellet. At block 186, the supernatant may be removed and the zBLT including the nucleic acid sample 166 may be suspended in a desired buffer to continue the biochemical manipulation reaction. At least in some instances, unbinding of the nucleic acid may be facilitated via treatment of the zBLT 70a bound to the nucleic acid sample 166 with a surfactant (e.g., an anionic detergent such as sodium dodecyl sulfate (SDS)) or treatment with ethylenediaminetetraacetic acid (EDTA) to chelate the Mg²⁺ cofactor.

As another example of a technique for performing buffer exchange using the zBLT 70a, FIG. 19C shows a flow diagram of a method 190 for performing buffer exchange after biochemical manipulation. At block 192, the vessel 164 including the nucleic acid sample 166 may be provided with the enzyme 168 and the adaptor 170 for performing the biochemical manipulation reaction. After the adaptor 170 is added to the nucleic acid sample 166, the zBLT 70a may be added. Subsequently, the nucleic acid sample 166 including the adaptor 170 and bound to the zBLT 70a may be pelleted via magnetic separation. At block 196, the supernatant may be removed and the zBLT including the nucleic acid sample 166 including the adaptor 170 may be suspended in a desired buffer.

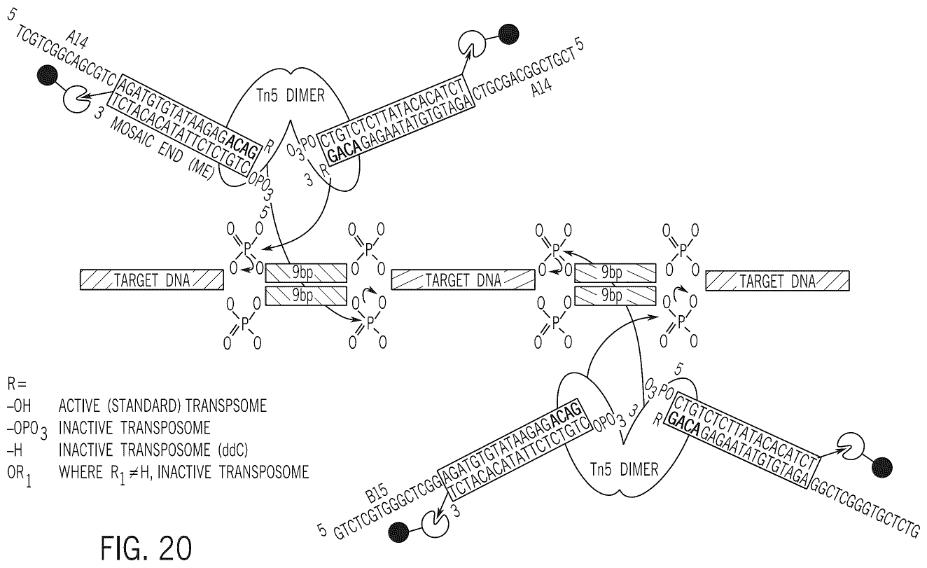

As discussed herein, an inactive transposome refers to a catalytically inactive transposome that is unable to join (e.g., insert via ligation) into a target nucleic acid, but the inactive transposome may still bind to the target nucleic acid. At least in some instances, the inactive transposome may be inactive due to inactivation of the transposase of the transposome, such as by modifying an amino acid sequence of the transposase. In some embodiments, the transposome may be in inactive due to modifications of oligonucleotides forming adaptors of the transposome that render the transposome inactive, while the transposase may still be active. To illustrate an example of an inactive transposome, FIG. 20 shows a schematic diagram illustrating a mechanism of binding between a transposome and a target nucleic acid.

During transposition, Tn5 transposase facilitates the nucleophilic attack of the 3′ hydroxyl group of the mosaic end (ME) transferred strand (TS) on the phosphodiester backbone of the target DNA, leading to attachment of the transferred strand to the substrate DNA (FIG. 1). Certain methods to inactivate enzymatic activity involve mutagenesis and protein engineering efforts. Techniques in accordance with the present disclosure include blocking of the 3′ hydroxyl group of the ME TS to block this chemical reactivity, while maintaining the ability to form dimeric transposomes and for the resulting transposome to bind DNA. An example of sequences to form such an inactivated transposome (i.e., inactive transposome 28), or “Zombie transposome”, specifically, as shown in the illustrated embodiment, by blocking the 3′ hydroxyl group of the transferred strand with a phosphate group, are shown in Table 2. It should be noted that alternative blocking groups may be used in place of the phosphate group, such as an ester, sulfate, a nitrate, carboxyl groups, or dideoxyCytosine (ddC), and the ddC may remove the oxygen at 3′ position. In the table, /3Phos/ and /5Phos/ refer to 3′ and 5′ phosphate groups, respectively. /3BiotinN/ refers to a 3′ biotin for linking the transposome to a streptavidin-coated magnetic bead.

TABLE 2
Oligonucleotide sequences for preparation of
inactive transposomes.

| Oligo Name | Oligo Sequence (5′ - 3′) |
|---|---|
| Inactive TS | AGATGTGTATAAGAGACAG/3Phos/ |
| ME′-biotin | /5Phos/CTGTCTCTTATACACATCT/3BiotinN/ |

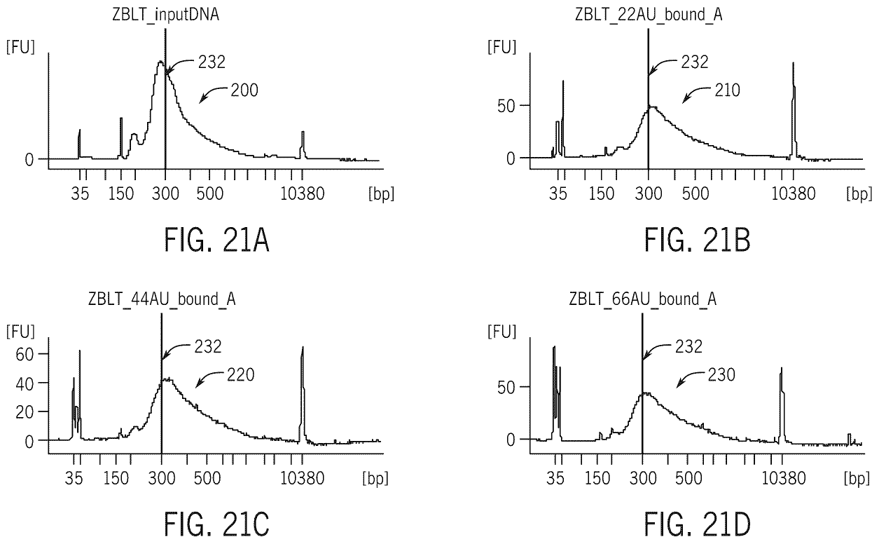

As described with respect to FIGS. 15A-15C, tuning the concentration or density (e.g., AU/μL) of transposomes (e.g., the inactive transposomes 28) may adjust the size-selectively of an SLTC 68 (e.g., the size of nucleic acid fragments that bind to the SLTC 68). In some embodiments, the density may be predetermined amount or range, such as between 10 to 100 AU/μL, 10 to 70 AU/μL, 20 to 60 AU/μL, 30 to 50 AU/μL, less than 100 AU/μL, less than 80 AU/μL, less than 70 AU/μL, less than 60 AU/μL, less than 50 AU/μL, less than 30 AU/μL, and so on. Further, the zSLTC 68 (e.g., a BLT 70 that does not include catalytically active transposomes) may be used to normalize nucleic acid fragments. As such, tuning the density of inactive transposomes 28 of zSLTCs 68 used for normalization may tune the size and/or amount of resulting normalized acid fragments. FIGS. 21A, 21B, 21C, and 21D generally illustrate that tuning the amount of inactive transposomes 28 bound to zSLTCs on a substrate 56 may tune the fragment size-selectivity of the zBLTs 70a. For example, FIG. 21A shows a graph 200 illustrating a distribution of nucleic acid fragment sizes of nucleic acids. The distribution of nucleic acid fragment sizes may be produced from a tagmentation reaction, as described herein. FIG. 21B shows a graph 210 illustrating a distribution of nucleic acid fragment sizes bound to beads 56 having a density of approximately 22 AU/μL. FIG. 21C shows a graph 220 illustrating a distribution of nucleic acid fragment sizes bound to beads 56 having a density of approximately 44 AU/μL. FIG. 21D shows a graph 230 illustrating a distribution of nucleic acid fragment sizes bound to beads 56 having a density of approximately 66 AU/μL. In each of FIGS. 21A-21D, a line 232 is shown at approximately 300 basepairs (bps) to illustrate the shift of nucleic acid fragment sizes bound to the zSLTC 68. As shown, the average fragment size of nucleic acid fragments bound to the zSLTC 68 increases with increasing density of inactive transposomes 28 bound to the beads 56. Accordingly, a system may include zSLTCs 68 generated to preferentially bind a certain size or distribution of nucleic acid fragments by tuning the density of inactive transposomes 28 on a zSLTC 68.

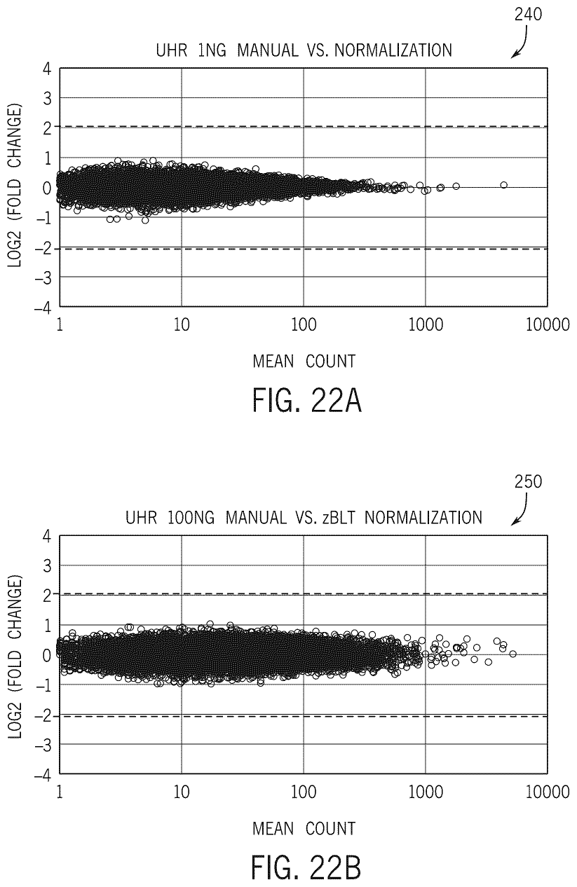

FIGS. 22A and 22B illustrate graphs of gene expression analysis results, which demonstrates that normalization with zSLTCs 68 show little to no difference in the expression of genes of nucleic acids as compared to conventional normalization techniques (e.g., manual normalization). In particular, FIG. 22A shows a graph 240 of gene expression analysis results (e.g., R²=0.93) corresponding to a first amount (e.g., 1 nanogram (ng)) of universal human reference (UHR) RNA. FIG. 22B shows a graph 250 of gene expression analysis results (e.g., R²=0.96) corresponding to a second amount (e.g., 100 ng) of UHR RNA. In general, the graphs 240, 250 both show a fold change between −2 and 2, which indicates that the normalization with zSLTC 68 produces generally similar gene expression results. However, normalization with zSLTC 68 may be significantly faster than certain conventional normalization techniques. For example, normalization with zSLTCs 68 may take up to 80% less time than manual normalization.

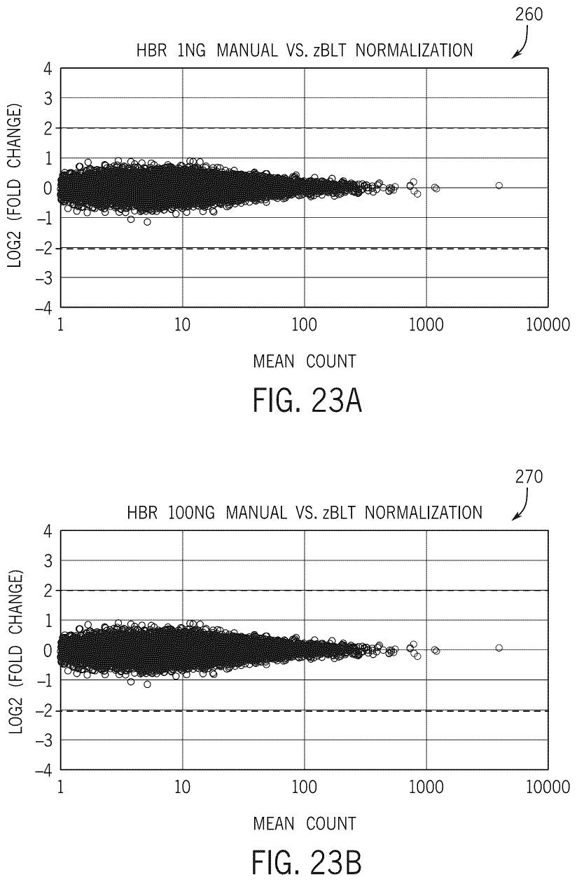

FIGS. 23A and 23B illustrate another example of graphs of gene expression analysis results. FIG. 23A shows a graph 260 of gene expression analysis results (e.g., R²=0.92) corresponding to a first amount (e.g., 1 nanogram (ng)) of human brain reference (HBR) RNA. FIG. 23B shows a graph 270 of gene expression analysis results (e.g., R²=0.96) corresponding to a second amount (e.g., 100 ng) of HBR RNA. In a generally similar manner as described in FIGS. 22A and 22B, the fold change indicates that the normalization with zSLTC 68 produces generally similar gene expression results as compared to the manual normalization. As such, the disclosed normalization techniques may improve the speed of normalizing nucleic acid fragments with little to no decrease in the quality of the results.

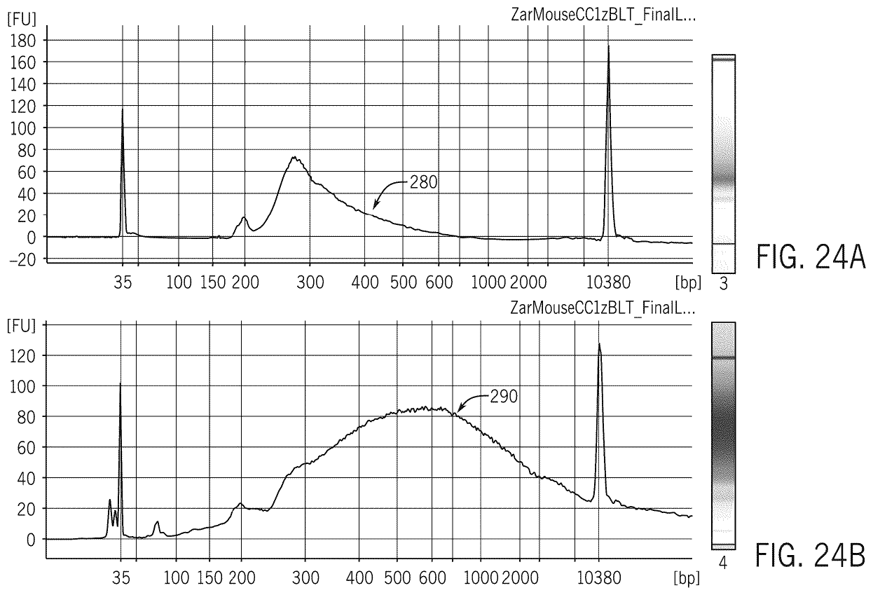

Accordingly, the disclosed techniques may enable tuning the size selectivity of zSLTCs 68 and improve the efficiency of normalization techniques. This is further illustrated in FIGS. 24A and 24B. More specifically, FIG. 24A shows a graph 280 of a distribution of nucleic acids (e.g., average size=346 bp) obtained using normalization techniques without zSLTC 68. FIG. 24B shows a graph 290 of a distribution of nucleic acids (e.g., average size=554 bp) obtained using normalization techniques with zSLTC 68. In general, FIG. 24B demonstrates that large nucleic acid fragments may be obtained using zBLTs. In some embodiments, the distribution of nucleic acids may more closely match the distribution of nucleic acids from the source. As such, normalization using zSLTC 68 may enable a user to capture more nucleic acids during normalization, thereby providing a normalization technique with reduce waste (e.g., wasting fewer nucleic acids) and a tunable size selectivity.

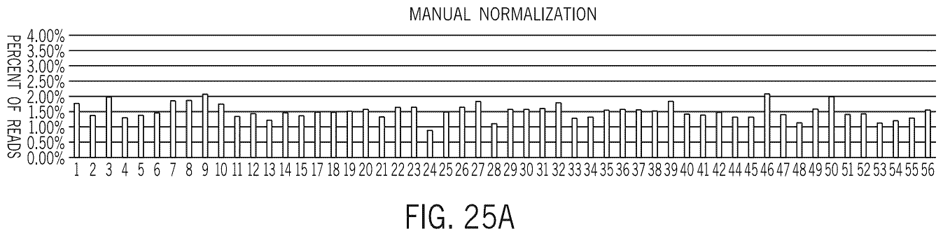

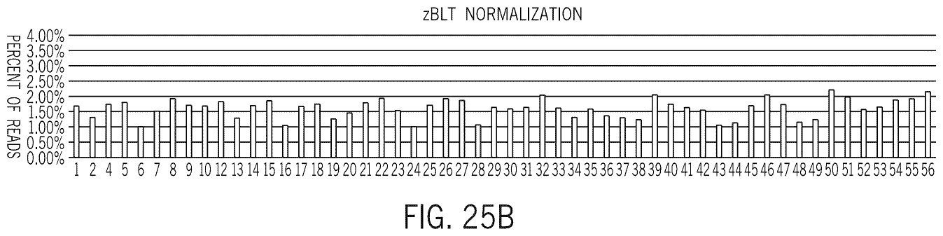

FIG. 25A shows a graph of reads mapping to each indexed sample (e.g., ‘1’, ‘2’, ‘3’, and so on represent different samples) versus the percentage corresponding to each sample obtained with manual normalization. FIG. 25B shows a graph of reads mapping to each sample versus the percentage of the corresponding samples obtained with zBLTs 70a. In general, FIGS. 25A and 25B demonstrate that normalization using zBLTs 70a produce a distribution of nucleic acid fragments that is comparable to manual normalization.

Accordingly, additional aspects of the present disclosure relate to a SLTC or BLT that includes active transposomes 30 and/or inactive transposomes 28. As discussed above, a BLT that includes active transposomes may produce fragments of a target nucleic acid via a tagmentation reaction occurring between the active transposomes and the target nucleic acid. As discussed herein, chemically inactivating a transposome (e.g., via addition of 3′ phosphate to the transferred strand) may not disrupt binding of the ME. Further, the inactivation may not it inhibit dimeric transposome formation or prevent binding of target DNA, despite the location of this modification within the active site of the complex. Furthermore, the sizes of the fragments of the target nucleic acid may be tuned by varying the number of active transposomes 30 and inactive transposomes 28 and/or the ratio of the number of active transposomes 30 and inactive transposomes 28 that are bound or linked to the substrate 56 of the SLTC 68 (e.g., BLT 72). Certain techniques may utilize a size-selecting SPRI to narrow the fragment size distribution to the desired range for sequencing, and such techniques may reduce the library conversion efficiency through discarding unwanted fragment sizes. The disclosed techniques provide control of insert size at the tagmentation step thereby reducing additional steps downstream such as size selection. Further still, the SLTC 68, such as the zBLT 70a, may be used for applications including separation of nucleic acids, normalizing an amount of nucleic acid between different samples, and performing buffer transfer.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for (perform)ing (a function) . . . ” or “step for (perform)ing (a function) . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to enable any person skilled in the art to practice the disclosed embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A transposome complex comprising:
a plurality of inactive transposomes coupled to one another;
a first active transposome coupled to a first end of the plurality of inactive transposomes;
a second active transposome coupled to a second end of the plurality of inactive transposomes such that the plurality of inactive transposomes are positioned between the first active transposome and the second active transposome; and
wherein the first active transposome, the second active transposome, and each inactive transposome of the plurality of inactive transposomes comprise a transposase and an adaptor.

2. The transposome complex of claim 1, wherein the first active transposome and the second active transposome are on opposing terminal ends of the transposome complex.

3. The transposome complex of claim 1, wherein the first active transposome and the second active transposome further comprise an insertion sequence.

4. The transposome complex of claim 1, wherein the first active transposome, the second active transposome, and each inactive transposome of the plurality of inactive transposomes is a dimer.

5. The transposome complex of claim 1, wherein each inactive transposome of the plurality of inactive is configured to bind to target nucleic acid and is inactive such that the adaptor is not inserted into target nucleic acid when bound.

6. The transposome complex of claim 5, wherein each inactive transposome is inactive based on a modification of the adaptor.

7. The transposome complex of claim 6, wherein the modification is a blocked 3′-end of the adaptor to remove catalytic activity.

8. The transposome complex of claim 6, wherein the modification is a dephosphorylation of a 5′-end of the adaptor to remove catalytic activity.

9. The transposome complex of claim 1, wherein the first active transposome and the second active transposome form catalytically active ends of the transposome complex.

10. The transposome complex of claim 1, wherein the plurality of inactive transposomes are coupled together via complementary adaptor sequences.

11. The transposome complex of claim 1, wherein the first active transposome comprises a first active transposase and a first adaptor that is different than the adaptor of each inactive transposome.

12. The transposome complex of claim 11, wherein the second active transposome comprises a second active transposase and a second adaptor that is different than the adaptor of each inactive transposome.

13. The transposome complex of claim 1, wherein each inactive transposome is inactive based on a modification to an amino acid sequence of a transposase of each inactive transposome.

14. The transposome complex of claim 1, wherein the plurality of inactive transposomes, the first active transposome, and the second active transposome are homodimers.

15. The transposome complex of claim 1, wherein the adaptor is at least partially double stranded, and wherein each inactive transposome of the plurality of inactive transposomes comprises a second adapter, wherein the e adapter and the second adaptor are the same.

16. The transposome complex of claim 15, wherein the adaptor comprises an at least partially double-stranded first adaptor sequence and wherein the second adaptor sequence coupled to a second transposase of the plurality of inactive transposomes comprises an at least partially double-stranded second adaptor sequence, wherein the first adaptor sequence and the second adaptor sequence on an individual inactive transposome of the plurality of inactive transposomes are the same.

17. The transposome complex of claim 1, wherein a first adaptor of the first active transposome and a second oligonucleotide adaptor of the second active transposome each comprise a double-stranded transposon end sequence and an at least partially double-stranded adaptor sequence.

18. The transposome complex of claim 1, wherein each inactive transposome is coupled to a neighboring transposome of the transposome complex via crosslinking.

19. The transposome complex of claim 1, where at least one inactive transposome includes a stabilizer configured to reduce monomeric exchange between at least one inactive transposome of the plurality of inactive transposomes and the first active transposome, the second active transposome, or both.

20. A kit comprising a plurality of the transposome complex of claim 1, wherein each transposome complex of the plurality has a same number of inactive transposomes between the the first active transposome and the second active transposome.

21. A method of preparing a transposome complex, comprising:
providing an initiator transposome comprising a first oligonucleotide adaptor and a second oligonucleotide adaptor; and
hybridizing at least one linking transposome to the initiator transposome via a linking adaptor of the at least one linking transposome, wherein the at least one linking transposome is catalytically inactive, and wherein the linking adaptor is complementary to the first oligonucleotide adaptor, the second oligonucleotide adaptor, or both; and
coupling at least one terminal transposome to the at least one linking transposome via a terminal adaptor of the terminal transposome that is complementary to the linking adaptor or a different linking adaptor of the at least one linking transposome, wherein the terminal transposome is catalytically active.

22. The method of claim 21, wherein the initiator transposome is an active transposome such that the initiator transposome may join to a target nucleic acid.

23. The method of claim 21, wherein the initiator transposome is an inactive transposome such that the initiator transposome is prevented from joining to a target nucleic acid.

24. The method of claim 21, comprising attaching the initator transposome to a substrate surface.

25. The method of claim 21, hybridizing at least two linking transposomes to opposing sides of the initiator transposome via a respective linking adaptor of the at least two linking transposome, wherein the respective linking adaptor is complementary to the first oligonucleotide adaptor and the second oligonucleotide adaptor.

26. The method of claim 21, wherein the first oligonucleotide adaptor and the second oligonucleotide adaptor comprise different nucleic acid sequences.

27. The method of claim 21, comprising washing a substrate containing the hybridized linking transposome and the initator transposome after hybridizing the at least one linking transposome to the initiator transposome via the linking adaptor of the at least one linking transposome.

28. The method of claim 21, wherein the at least one linking transposome comprises a plurality of linking transposomes, and wherein the coupling of the terminal comprises coupling to a different linking transposome than is hybridized to the initiator.

29. A method of preparing a nucleic acid library, comprising:
contacting target nucleic acids with a plurality of transposome complexes, wherein each transposome complex of the plurality comprises a first active transposome coupled to a second active transposome via an intervening plurality of inactive transposomes, to permit binding of the plurality of transposome complexes to the target nucleic acids; and
tagmenting the target nucleic acids to generate nucleic acid fragments, wherein a size of the generated nucleic acid fragments is a function of a size of an individual transposome complex of the plurality of transposome complexes.

30. The method of claim 29, further comprising digesting regions of the target nucleic acids that are not bound by the plurality of transposome complexes.

31. The method of claim 29, further comprising removing the plurality of transposome complexes after generating the nucleic acid fragments.

32. The method of claim 29, further comprising sequencing the generated nucleic acid fragments.

33. The method of claim 29, wherein the plurality of transposome complexes all have about a same number of intervening inactive transposomes between the first active transposome and the second active transposome such that the generated nucleic acid fragments are within a size range.

34. The method of claim 29, wherein each transposome complex of the plurality of transposome complexes is bound to a respective substrate.

35. A surface-linked transposome complex comprising:
a solid surface; and
a plurality of transposomes coupled to the solid surface, and wherein at least one transposome of the plurality of transposomes is inactive based on a modification of an oligonucleotide adaptor of the at least one transposome.

36. The transposome complex of claim 35, wherein the oligonucleotide adaptor comprises a blocked 3′-end to remove catalytic activity.

37. The transposome complex of claim 36, wherein 3′ end is blocked via a phosphate group, dideoxyCytosine, an ester, a sulfate, a carboxyl group, or any combination thereof.

38. The transposome complex of claim 35, wherein each transposome of the plurality of transposomes is configured to bind to target nucleic acid and is inactive such that the oligonucleotide adaptor is not inserted into target nucleic acid when bound.

39. The transposome complex of claim 35, wherein the solid surface is a magnetic bead.

40. The transposome complex of claim 35, wherein the solid surface is planar substrate.

41. The transposome complex of claim 35, wherein each transposome of the plurality of transposomes is coupled to the solid surface via a linker.

42. The transposome complex of claim 35, wherein the plurality of transposomes are at a regular distance from one another on the solid surface.

43. The transposome complex of claim 35, comprising a nucleic acid bound to at least a portion of the plurality of transposomes.

44. The transposome complex of claim 43, wherein the nucleic acid is a double-stranded nucleic acid.

45. The transposome complex of claim 35, wherein the solid surface is not coupled to any active transposomes.

46. A method of separating nucleic acids, comprising:
contacting a plurality of inactive transposome complexes with a mixed nucleic acid sample in solution, the mixed nucleic acid sample comprising double-stranded DNA and RNA such that the double-stranded DNA selectively binds to the plurality of inactive transposome complexes relative to the RNA, wherein each inactive transposome complex of the plurality comprises a plurality of inactive transposomes coupled to a surface to permit binding of the plurality of inactive transposome complexes to the double-stranded DNA;
and
separating the double-stranded DNA from RNA by removing the plurality of inactive transposomes complexes with bound double-stranded DNA from the solution, the solution comprising the RNA.

47. The method of claim 46, comprising increasing a concentration of Mg²⁺ of the solution to facilitate binding of the double-stranded DNA to the plurality of inactive transposomes complexes.

48. The method of claim 46, comprising separating the bound double-stranded DNA from the plurality of inactive transposomes complexes by eluting the double-stranded DNA into a second solution.

49. The method of claim 46, wherein the surface comprises a magnetic bead, and wherein the separating comprises magnetic separation.

50. A method of normalizing an amount of nucleic acids for a plurality of samples, comprising:
contacting a first plurality of double-stranded nucleic acids of a first sample with a first plurality of inactive transposome complexes, wherein each inactive transposome complex of the first plurality of transposome complexes comprises a predetermined amount of inactive transposomes coupled to a bead surface, and wherein the contacting is under conditions such that a portion of the first plurality of double-stranded nucleic acids binds to the first plurality of inactive transposome complexes;
contacting a second plurality of double-stranded nucleic acids of a second sample with a second plurality of inactive transposome complexes, wherein each inactive transposome complex of the second plurality of inactive transposome complexes comprises the predetermined amount of inactive transposomes coupled to a bead surface, and wherein the contacting is under conditions such that a portion of the second plurality of double-stranded nucleic acids binds to the second plurality of inactive transposome complexes; and
sequencing the bound portion of the first plurality of double-stranded nucleic acids and the bound portion of the second plurality of double-stranded nucleic acids.

51. The method of claim 50, comprising separating the bound portion of the first plurality of double-stranded nucleic acids from unbound nucleic acids in the first sample prior to sequencing.

52. The method of claim 51, comprising separating the bound portion of the second plurality of double-stranded nucleic acids from unbound nucleic acids in the second sample prior to sequencing.

53. The method of claim 51, wherein the bound portion of the first plurality of double-stranded nucleic acids and the bound portion of the second plurality of double-stranded nucleic acids are about a same amount of nucleic acid relative to one another.

54. The method of claim 51, wherein the predetermined range of inactive transposomes coupled to the bead surface is between approximately 10 AU/μL to approximately 70 AU/μL.

55. The method of claim 51, wherein the predetermined range of inactive transposomes coupled to the bead surface is between approximately 20 AU/μL to approximately 60 AU/μL.

56. A method of performing a buffer exchange, comprising:
contacting a plurality of nucleic acids suspended in a first buffer solution with a plurality of inactive transposome complexes, wherein each inactive transposome complex of the plurality of inactive transposome complexes comprises a plurality of inactive transposomes coupled to a surface;
producing a pellet comprising the plurality of nucleic acids bound to the plurality of inactive transposome complexes;
separating the pellet from the first buffer solution; and
suspending the pellet in a second buffer solution.

57. The method of claim 56, comprising washing the pellet.
